(19) United States
(12) Patent Application Publication
(10) Pub. No.: US 2016/0251441 A1
(43) Pub. Date: Sep. 1, 2016

(54) ANTIBODY PURIFICATION

(71) Applicant: MEDIMMUNE, LLC, Gaithersburg, MD (US)

(72) Inventors: Ellen T. O'CONNOR, Gaithersburg, MD (US); Mutsa Y. KAMBARAMI, Gaithersburg, MD (US); Matthew T. ASPELUND, Gaithersburg, MD (US); Frank L. BARTNIK, Gaithersburg, MD (US); Mark BERGE, Gaithersburg, MD (US); Thoa BUI, Gaithersburg, MD (US); Methal ALBARGHOUTHI, Gaithersburg, MD (US); Jihong WANG, Gaithersburg, MD (US); Jun KIM, Gaithersburg, MD (US)

(73) Assignee: MedImmune, LLC, Gaithersburg, MD (US)

(21) Appl. No.: 15/028,897

(22) PCT Filed: Oct. 23, 2014
(86) PCT No.: PCT/US2014/061890

§ 371 Date: Apr. 12, 2016

Related U.S. Application Data
(60) Provisional application No. 61/895,625, filed on Oct. 25, 2013.

(51) Int. Cl. C07K 16/28; C07K 16/06; B01D 15/38; C07K 1/16

(52) U.S. Cl. CPC C07K 16/2866; C07K 1/165; C07K 16/065; B01D 15/3847; C07K 2317/565; C07K 2317/21

(57) ABSTRACT

Described herein is a method for separating antibody fragment impurities from target antibodies or desired fragments thereof.

### 1. INTRODUCTION

### 1.1. Reference to a Sequence Listing

This application incorporates by reference a Sequence Listing submitted with this application as text file IFNAR-450WO1_SL.txt created on Oct. 21, 2014, and having a size of 4 kilobytes.

### 1.2. Field of the Invention

The present invention relates to the purification of recombinantly produced proteins. In a more particular embodiment, the invention relates to methods of separating antibody fragment impurities from a target antibody or a desired fragment thereof.

### 1.3. Background of the Invention

Recombinantly produced polypeptides, such as antibodies, are used in a wide array of diagnostic and therapeutic applications. The process of manufacturing recombinant polypeptides, such as antibodies, generally involves expression of the polypeptide in a host cell (also known as “upstream processing”), and purification of the polypeptide (also known as “downstream processing”).

Expression generally involves culturing a prokaryotic or eukaryotic host cell under appropriate conditions for the host cells to produce the antibody. The increased demand for antibody therapeutics has driven the development of efficient upstream processes with ever increasing titers, with titers of 10 g/L obtained during cell culture.

Once a recombinant antibody is expressed, intact host cells and cell debris are separated from the cell culture media in a process referred to as “cell harvesting.” For example, host cells can be separated from the cell culture media by centrifugation or filtration to provide a clarified fluid (which can be referred to as the “cell culture supernatant”) that includes the target antibody and other impurities. Examples of impurities that may be found in the clarified cell culture supernatant include, but are not limited to, host cell proteins (HCP), nucleic acids, endotoxins, viruses, protein variants, protein aggregates and antibody fragment impurities.

Purification refers to the removal of impurities from the clarified cell culture supernatant and typically involves one or more chromatography steps. Typical processes include capture, intermediate purification or polishing, and final polishing steps. Protein A Affinity chromatography, for example, or ion exchange chromatography, is often used as a capture step. Often, capture is followed by at least two intermediate purification or polishing steps to increase purity and remove viral contaminants. Intermediate purification or polishing steps are often accomplished by affinity chromatography, ion exchange chromatography, or hydrophobic interaction chromatography (HIC). In many processes, the final polishing step is accomplished using ion exchange chromatography, hydrophobic interaction chromatography, or gel filtration.

The increases in upstream titers achieved in cell culture processes, while desirable for productivity, can present additional challenges for downstream processes. For example, increased titer can result in increased levels of impurities, such as aggregate, HCP, DNA, and antibody fragment impurities. Although methods are known for optimizing platform downstream processes for removal of aggregate, HCP and DNA impurities, downstream platform processes have not historically been designed for the removal of antibody fragment impurities.

Few studies have examined methods and mechanisms of monoclonal antibody fragment separation. Accordingly, little is known about separation of IgG fragment impurities, such as large hinge, small hinge, heavy-heavy-light, heavy-light, light-light, heavy-heavy, heavy, and light. As such, there remains a need for purification processes that efficiently remove impurities, such as antibody fragment impurities.

### 2. SUMMARY OF THE INVENTION

In one embodiment, a method for separating antibody fragmentation product impurities from a target antibody or a desired antigen-binding fragment thereof is provided, wherein the method includes: providing a starting material including the target antibody or desired antigen-binding fragment, antibody fragmentation product impurities and a loading buffer; loading the starting material on a mixed mode chromatography column; allowing the material to flow through the column, wherein at least some of the antibody fragmentation product impurities are adsorbed to a stationary phase of the mixed mode chromatography resin and at least some of the target antibody or desired antigen-binding fragment thereof is eluted from the column in one or more eluent fractions; and recovering one or more eluent fractions, wherein one or more eluent fractions are enriched in the target antibody or desired antigen-binding fragment thereof as compared to the starting material. In another embodiment, a method of decreasing antibody fragmentation product impurities in a composition that includes target antibody or desired antigen-binding fragment thereof is provided.

In one embodiment, the mixed mode chromatography column employs separation mechanisms selected from: anion exchange (AEX), cation exchange (CEX), hydrophobic interaction (HIC), hydrophilic interaction, hydrogen bonding, pi-pi bonding and metal affinity. In another embodiment, the mixed mode chromatography column employs a combination of AEX and HIC interactions. In a more particular embodiment, the mixed mode chromatography column includes a Capto Adhere™ mixed mode chromatography column.

In one embodiment, the starting material includes between about 88% and about 98% target antibody or desired antigen-binding fragment thereof. In one embodiment, the starting material includes up to about 98% target antibody or desired antigen-binding fragment thereof. In one embodiment, one or more eluent fractions include between about 98% and 99.9% target antibody or desired antigen-binding fragment thereof. In one embodiment, one or more eluent fractions include at least about 98% target antibody or desired antigen-binding fragment thereof. In one embodiment, one or more eluent fractions include between about 1% and about 10% more target antibody or desired antigen-binding fragment thereof than the starting material. In one embodiment, one or more eluent fractions include between about 1% and about 3% more target antibody or desired antigen-binding fragment thereof than the starting material.

In one embodiment, the starting material includes between about 1% and about 10% antibody fragmentation product impurities. In one embodiment, the starting material includes between about 1% and about 3% antibody fragmentation product impurities. In one embodiment, one or more eluent fractions include less than about 3% antibody fragmentation product impurities. In one embodiment, one or more eluent fractions include less than about 2% antibody fragmentation product impurities. In one embodiment, one or more eluent fractions include less than about 1% antibody fragmentation product impurities.

In one embodiment, the pH of the starting material is between about 5 and about 8. In one embodiment, the pH of the starting material is between about 5.5 and about 6.5. In one embodiment, the pH of the starting material is between about 5.7 and about 6.0. In one embodiment, the starting material has a conductivity of between about 0 mS/cm and about 30 mS/cm. In one embodiment, the starting material has a conductivity of less than about 10 mS/cm. In one embodiment, the starting material has a conductivity of less than about 7 mS/cm.

In one embodiment, the starting material has a load capacity of between about 75 g/L and about 250 g/L. In one embodiment, the starting material has a load capacity of between about 75 g/L and about 125 g/L.

In one embodiment, the target antibody or desired antigen-binding fragment thereof includes a light chain sequence having an amino acid sequence shown in SEQ ID NO:2. In one embodiment, the target antibody or desired antigen-binding fragment thereof includes a heavy chain sequence having an amino acid sequence shown in SEQ ID NO:1. In one embodiment, the target antibody or desired antigen-binding fragment thereof includes a light chain sequence having an amino acid sequence shown in SEQ ID NO:2 and a heavy chain sequence having an amino acid sequence shown in SEQ ID NO:1. In one embodiment, the target antibody or desired antigen-binding fragment thereof includes one or more light chain CDR amino acid sequences selected from LCDR1, LCDR2, LCDR3 of the light chain amino acid sequence shown in SEQ ID NO: 2, and combinations thereof. In one embodiment, the target antibody or desired antigen-binding fragment thereof includes one or more heavy chain CDR amino acid sequences selected from HCDR1, HCDR2, HCDR3 of the heavy chain amino acid sequence shown in SEQ ID NO:1, and combinations thereof. In one embodiment, the target antibody or desired antigen-binding fragment thereof includes one or more light chain CDR amino acid sequences selected from LCDR1, LCDR2, LCDR3 of the light chain amino acid sequence shown in SEQ ID NO:2, and combinations thereof, and one or more heavy chain CDR amino acid sequences selected from HCDR1, HCDR2, HCDR3 of heavy chain amino acid sequence shown in SEQ ID NO:1, and combinations thereof.

In one embodiment, the loading buffer has a conductivity of less than about 10 mS/cm. In one embodiment, the loading buffer has a conductivity of less than about 4 mS/cm.

In one embodiment, the loading buffer is selected from phosphate buffer, citrate buffer, sodium acetate, histidine, MOPS, HEPES, TRIS and combinations thereof. In one embodiment, the loading buffer is selected from sodium phosphate, potassium phosphate, sodium citrate, and combinations thereof. In one embodiment, the loading buffer has a concentration of between about 1 mM and about 50 mM.

In one embodiment, a composition including a target antibody or desired antigen-binding fragment is provided, wherein the composition includes at least about 99% target antibody or desired antigen-binding fragment thereof and less than about 2% antibody fragmentation product impurity. In one embodiment, the composition includes at least about 99% target antibody or desired antigen-binding fragment thereof and no more than 1% antibody fragmentation binding product. In one embodiment, the composition includes at least about 99.5% target antibody or desired antigen-binding fragment thereof and no more than about 0.5% antibody fragmentation binding product. In one embodiment, the composition includes no more than about 3% aggregate. In one embodiment, the composition includes no more than about 2.5% aggregate.

In one embodiment, the antibody fragmentation product impurities include peptide cleavage fragments. In one embodiment, the antibody fragmentation product impurities are selected from heavy chain fragmentation products, hinge region fragmentation products, light chain fragmentation products, and combinations thereof. In one embodiment, the antibody fragmentation product impurities include antibody reduction fragments. In one embodiment, the antibody fragmentation product impurities are IgG fragments selected from heavy-heavy-light, heavy-light, light-light, heavy-heavy, heavy, and light.

In one embodiment, the target antibody or desired antigen-binding fragment thereof includes a light chain sequence having an amino acid sequence shown in SEQ ID NO:2. In one embodiment, the target antibody or desired antigen-binding fragment thereof includes a heavy chain amino acid sequence having an amino acid sequence shown in SEQ ID NO:1. In one embodiment, the target antibody or desired antigen-binding fragment thereof includes a light chain sequence having an amino acid sequence shown in SEQ ID NO:2 and a heavy chain amino acid sequence having an amino acid sequence shown in SEQ ID NO:1. In one embodiment, the target antibody or desired antigen-binding fragment thereof includes one or more light chain CDR amino acid sequences selected from LCDR1, LCDR2, LCDR3 of the light chain amino acid sequence shown in SEQ ID NO:2, and combinations thereof. In one embodiment, the target antibody or desired antigen-binding fragment thereof includes one or more heavy chain CDR amino acid sequences selected from HCDR1, HCDR2, HCDR3 of the heavy chain amino acid sequence shown in SEQ ID NO:1, and combinations thereof. In one embodiment, the target antibody or desired antigen-binding fragment thereof includes one or more light chain CDR amino acid sequences selected from LCDR1, LCDR2, LCDR3 of the light chain amino acid sequence shown in SEQ ID NO:2, and combinations thereof, and one or more heavy chain CDR amino acid sequences selected from HCDR1, HCDR2, HCDR3 of the heavy chain amino acid sequence shown in SEQ ID NO:1, and combinations thereof.

### 3. BRIEF DESCRIPTION OF THE FIGURES

FIGS. 1A and 1B are diagrammatic representations of exemplary monoclonal antibody fragmentation products.

FIG. 2 is a graph showing the impact of pH on fragment removal and yield.

FIGS. 3A and 3B show Reverse-Phase Chromatography (RPLC) profiles for purification at pH6.

FIGS. 4A and 4B show High Pressure Size Exclusion Chromatography (HPSEC) profiles for purification at pH6.

FIG. 5 shows the impact of conductivity and load capacity on purity at pH ranges near pH 6.

FIG. 6 shows the product quality for a scale up operation as determined by HPSEC.

FIG. 7 shows the product quality for a scale up operation as determined by RPLC.

FIG. 8 shows the impact of spiking the load with additional antibody fragmentation product impurity before purification.

FIG. 9 is a schematic representation of the (A) Capto Adhere™ ligand, (B) Capto Q™ ligand, and (C) Capto Phenyl™ ligand.

FIG. 10 shows the results for purification of a low pH viral inactivated material using Capto Q™ Resin.

FIG. 11 shows the results for purification of a low pH viral inactivated material using Capto Phenyl™ Resin.

FIG. 12 is a flow chart for a representative purification process for a recombinantly produced antibody.

FIGS. 13A and 13B are Bioanalyzer chromatograms showing levels of HHL, HH or LHF, HL, H or SHF and L fragments during purification using Capto Adhere™ resin at pH 5, 5.5, 6, 6.5, 7, 7.5, and 8.

FIGS. 14A, 14B, and 14C are Bioanalyzer chromatograms showing levels of HHL, HH or LHF, HL, H or SHF and L fragments, as well as dimer during purification using Capto Adhere™ resin at pH ranging from 5 to 6, conductivity ranging from 0 to 30 mS/cm, and load capacity ranging from 75 to 300 g/L.

### 4. DETAILED DESCRIPTION

### 4.1. Introduction

A common problem encountered during antibody purification, particularly as upstream titers increase, is the presence of undesirable antibody fragment contaminants. The presence of antibody fragment impurities can be problematic, particularly for therapeutic monoclonal antibody products, because fragmentation can result in loss of stability, aggregation, batch-to-batch inconsistency and may result in adverse reactions upon administration. Whereas many platform downstream processes focus on removal of aggregate, HCP, DNA, and potential viral contaminants, processes which efficiently remove antibody fragmentation product impurities are needed. Described herein is a purification process for separating antibody fragmentation product impurities from recombinantly produced antibodies or desired antigen-binding fragments thereof.

### 4.2. Terminology

Unless otherwise defined, scientific and technical terms used herein shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, and protein and oligo- or polynucleotide chemistry and hybridization described herein are those well-known and commonly used in the art. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used in accordance with the present disclosure, the following terms, unless otherwise indicated, shall be understood to have the following meanings:

As used herein, the term “about” is used to modify, for example, the quantity of an ingredient in a composition, concentration, volume, process temperature, process time, yield, flow rate, pressure, and ranges thereof, employed in describing the invention. The term “about” refers to variation in the numerical quantity that can occur, for example, through typical measuring and handling procedures used for making compounds, compositions, concentrates or formulations; through inadvertent error in these procedures; through differences in the manufacture, source, or purity of starting materials or ingredients used to carry out the methods, and other similar considerations. The term “about” also encompasses amounts that differ due to aging of a formulation with a particular initial concentration or mixture, and amounts that differ due to mixing or processing a formulation with a particular initial concentration or mixture. Where modified by the term “about,” the claims appended hereto include such equivalents.

As used herein, the term “antibody” refers to a polypeptide or group of polypeptides that include at least one antigen-binding domain that is formed from the folding of polypeptide chains having three-dimensional binding spaces with internal surface shapes and charge distributions complementary to the features of an antigenic determinant of an antigen. An antibody typically has a tetrameric form, with two pairs of polypeptide chains, each pair having one “light” and one “heavy” chain. The variable regions of each light/heavy chain pair form an antigen-binding site. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains (CH). Each light chain has a variable domain at one end (VL) and a constant domain (CL) at its other end, wherein the constant domain of the light chain is aligned with the first constant domain of the heavy chain, and the light chain variable domain is aligned with the variable domain of the heavy chain. Light chains are classified as either lambda chains or kappa chains based on the amino acid sequence of the light chain constant region. The variable domain of a kappa light chain may also be denoted herein as VK.

The terms “antibody” and “antibodies” as used herein refer to immunoglobulin molecules and immunologically active fragments of immunoglobulin molecules, i.e., molecules that contain at least one antigen-binding site. Immunoglobulin molecules can be of any isotype (e.g., IgG, IgE, IgM, IgD, IgA and IgY), subisotype (e.g., IgG1, IgG2, IgG3, IgG4, IgA1 and IgA2) or allotype (e.g., Gm, e.g., G1m(f, z, a or x), G2m(n), G3m(g, b, or c), Am, Em, and Km(1, 2 or 3)). Antibodies may be derived from any mammalian species, including, but not limited to, humans, monkeys, pigs, horses, rabbits, dogs, cats, mice, etc., or other animals such as birds (e.g. chickens). In one embodiment, the antibody may be fused to a heterologous polypeptide sequence, for example, a tag to facilitate purification. “Antibodies” include, but are not limited to, monoclonal antibodies (including full-length monoclonal antibodies), polyclonal antibodies, multispecific antibodies formed from at least two different epitope binding fragments (e.g., bispecific antibodies), CDR-grafted, human antibodies, humanized antibodies, camelised antibodies, chimeric antibodies, anti-idiotypic (anti-Id) antibodies, intrabodies, and desirable antigen-binding fragments thereof, including recombinantly produced antibody fragments. As used herein, the term “desirable antibody fragments,” “desirable antigen-binding fragments” or “target antibody fragment” can refer to antibody fragments formed by selective cleavage (e.g., proteolytic digestion) of an antibody, wherein the antibody fragment exhibits a desired biological activity and also antibody fragments expressed by a host cell. In one embodiment, an antibody fragment is a therapeutic antibody fragment that includes the antigen binding portion of an antibody. Examples of antibody fragments that can be recombinantly produced include, but are not limited to, antibody fragments that include variable heavy- and light-chain domains, such as single-chain Fvs (scFv), single-chain antibodies, Fab fragments, Fab′ fragments, F(ab′)₂ fragments. Antibody fragments can also include epitope-binding fragments or derivatives of any of the antibodies enumerated above. The terms “target antibody fragments” or “target” and “recombinant antibody fragments” or “recombinantly produced antibody fragments,” as used herein, do not include antibody fragmentation product impurities as discussed below.

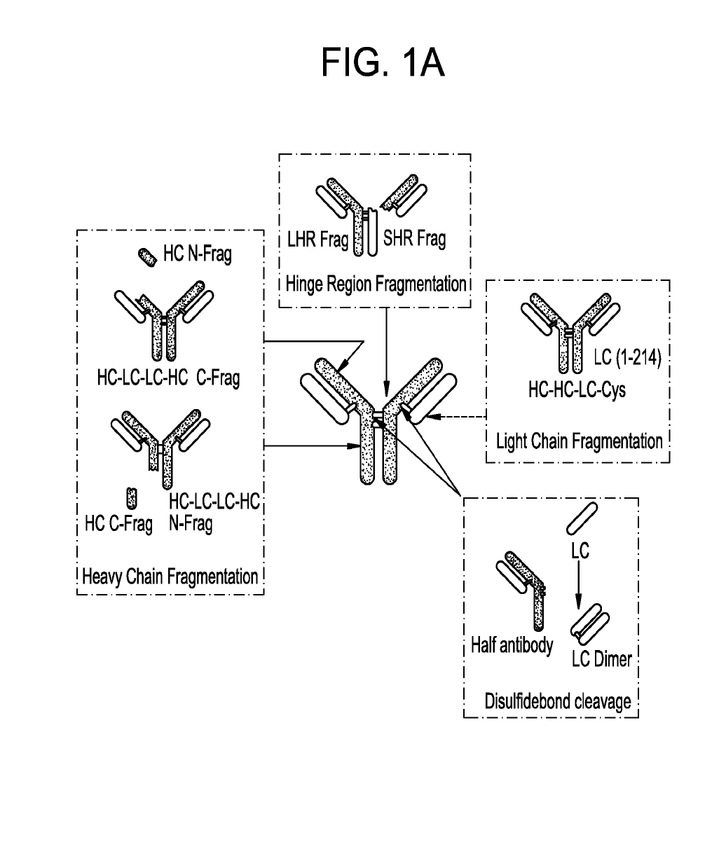

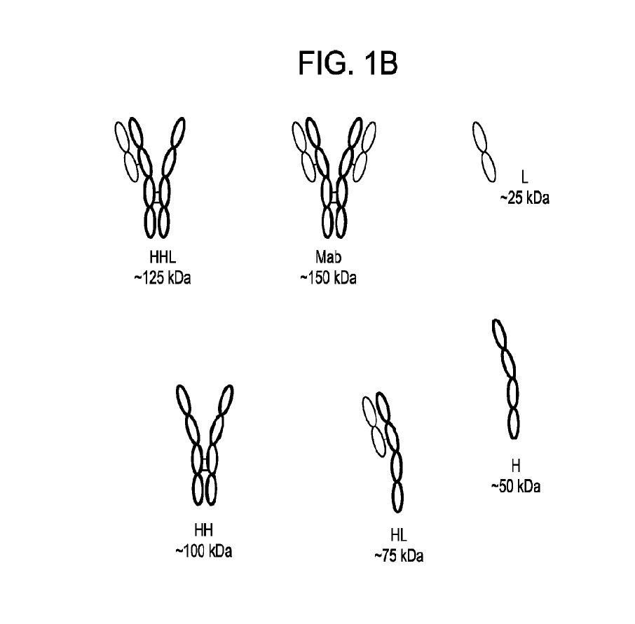

As used herein, the term “antibody fragmentation product impurities” refers to undesirable impurities that may be found in a composition that includes a target antibody or desired antigen-binding fragment thereof. Antibody fragmentation product impurities result from undesired and/or indiscriminate disruption of one or more covalent peptide bonds along the peptide backbone of a desired antibody product, which can result from non-enzymatic and/or enzymatic reactions. Unless specifically noted herein, the term “antibody fragmentation product impurities” does not refer to chemical modifications of side chains or disulfide bonds that do not disrupt the protein backbone. Antibody fragmentation product impurities can be formed by disruption of one or more covalent peptide bonds in various regions of the antibody, including, but not limited to the hinge region; one or more constant immunoglobulin domains; and one or more variable domains, for example, in one or more CDRs. Fragmentation in the hinge region that results in the generation of antibody fragments lacking Fc-mediated effector function can result in a reduced circulation half-time. Hinge fragmentation that results in an Fc-Fab fragment may result in an antibody product with reduced potency, particularly if the target receptor requires both Fab arms. Depending on the cleavage site, fragmentation in the constant region may have an effect on either the Fc-mediated effector function or on the circulation half-time. Fragmentation in the variable region is likely to have an effect on the binding of the antibody to the target and, consequently, may have an adverse effect on potency. FIGS. 1A and 1B provide diagrammatic representations of exemplary antibody fragmentation products.

The term “bind” or “binding” when discussing the interaction between a molecule and a column material means exposing the molecule to the column material under conditions such that the molecule is reversibly immobilized in or on the column material.

The term “cell culture supernatant” refers to a solution that is obtained by culturing host cells that produce a recombinant antibody of interest. In addition to the recombinant antibody, the cell culture supernatant may also include components of cell culture medium, metabolic byproducts secreted by the host cells as well as other components of the cultured cells. As used herein, the term “clarified cell culture supernatant” refers to a composition from which the host cells have been removed or harvested, such that the cell culture supernatant is generally free of cellular debris and/or intact cells.

The term “excipient” as used herein refers to an inert substance which is commonly used as a diluent, vehicle, preservative, binder or stabilizing agent for drugs which imparts a beneficial physical property to a formulation, such as increased protein stability, increased protein solubility, and/or decreased viscosity. Examples of excipients include, but are not limited to, proteins (for example, but not limited to, serum albumin), amino acids (for example, but not limited to, aspartic acid, glutamic acid, lysine, arginine, glycine), surfactants (for example, but not limited to, SDS, Tween 20, Tween 80, polysorbate and nonionic surfactants), saccharides (for example, but not limited to, glucose, sucrose, maltose and trehalose), polyols (for example, but not limited to, mannitol and sorbitol), fatty acids and phospholipids (for example, but not limited to, alkyl sulfonates and caprylate).

The phrase “host cell” or “host cells” refers to cells which express a recombinant polypeptide, for example, a recombinant antibody or recombinant antibody fragment. In particular, the term “host cell” refers to a cell that can or has taken up a nucleic acid, such as a vector, and supports replication of the nucleic acid and production of one or more encoded products. The term “host cell” can refer to a variety of cell types including prokaryotic cells, such as Escherichia coli, Lactococcus lactis and Bacillus species; yeast cells, such as Pichia pastoris, Pichia methanolica, and Saccharomyces cerevisiae; insect cell, such as bacuolovirus and eukaryotic cells. Examples of eukaryotic host cells include mammalian cells, for example, Chinese hamster ovary (CHO) cells, human embryonic kidney (HEK 293) cells, Vero cells, baby hamster kidney (BHK) cells, HeLa cells, CV1 monkey kidney cells, Madin-Darby Canine Kidney (MDCK) cells, 3T3 cells, myeloma cell lines, COS cells (e.g., COS1 and COS7) PC12, WI38 cells. The term host cell also encompasses combinations or mixtures of cells such as mixed cultures of different cell types or cell lines.

The term “impurity” refers to any foreign material, particularly a biological macromolecule such as DNA, RNA, or a protein, other than a target antibody or antibody fragment that is present in a sample. Contaminants can include antibody fragmentation product impurities.

The term “purify” or “purifying” a target antibody or desired fragment thereof from a composition or solution that includes the target antibody or desired fragment thereof and one or more contaminants means increasing the degree of purity of the target antibody or desired antibody fragment thereof in the composition or solution by removing (completely or partially) at least one contaminant from the composition or solution.

The term “mAb” refers to a monoclonal antibody.

The phrase “pharmaceutically acceptable” as used herein means approved by a regulatory agency of a Federal or state government, or listed in the U.S. Pharmacopeia, European Pharmacopia or other generally recognized pharmacopeia for use in animals, and more particularly in humans.

The terms “polypeptide” or “protein” can be used interchangeably to refer to a molecule having two or more amino acid residues joined to each other by peptide bonds. The term “polypeptide” can refer to antibodies and other non-antibody proteins. Non-antibody proteins include, but are not limited to, proteins such as enzymes, receptors, ligands of a cell surface protein, secreted proteins and fusion proteins or fragments thereof. The polypeptide may or may not be fused to another polypeptide. Polypeptides can also include modifications such as, but not limited to, glycosylation, lipid attachment, sulfation, gamma-carboxylation of glutamic acid residues, hydroxylation and ADP-ribosylation. Polypeptides can be of scientific or commercial interest, including protein-based therapeutics.

The term “recombinant” refers to a biological material, for example, a nucleic acid or protein, that has been artificially or synthetically (i.e., non-naturally) altered or produced by human intervention.

The term “remove,” when used in context of removal of antibody fragmentation product impurities, refers to decrease in the amount of antibody fragmentation product impurities in the purified product. Removal may or may not result in the absence of antibody fragmentation product impurities from the purified product. In general, removal refers to at least a 2 fold, 3 fold, 4 fold, 5 fold, 10 fold, 15 fold, 20 fold, 25 fold and up to 30 fold, 35 fold, 40 fold, 45 fold or 50 fold decrease in antibody fragmentation product impurities in the purified product when compared to the level of antibody fragmentation product impurities in the original composition.

The terms “stability” and “stable” as used herein in the context of a formulation of a recombinantly produced polypeptide, for example, a pharmaceutical formulation that includes a recombinantly produced antibody or antibody fragment, refer to the resistance of the polypeptide in the formulation to particle formation, aggregation, degradation or fragmentation under manufacture, preparation, transportation and storage conditions. A “stable” formulation retains biological activity under manufacture, preparation, transportation and storage conditions. Stability can be assessed by degrees of particle formation, aggregation, degradation or fragmentation, as measured by HPSEC, static light scattering (SLS), Fourier Transform Infrared Spectroscopy (FTIR), circular dichroism (CD), urea unfolding techniques, intrinsic tryptophan fluorescence, differential scanning calorimetry, and/or ANS binding techniques, as compared to a reference formulation.

As used herein, “substantially pure” refers to a target material that is the predominant species present (e.g., on a molar basis it is more abundant than any other individual species in the composition). In one embodiment, a substantially purified fraction is a composition wherein the target material includes at least about 50% (on a molar basis) of all macromolecular species present. Generally, a substantially pure composition will include more than about 80% target molecule as compared to all macromolecular species present in the composition, or more than about 85%, more than about 90%, more than about 95%, or more than about 99% target material. In one embodiment, the target material is purified to essential homogeneity (contaminant species cannot be detected in the composition by conventional detection methods) and the composition includes essentially a single macromolecular species, for example, the target macromolecule.

### 4.3. Recombinant Antibody Production

In one embodiment, a recombinant antibody is produced using host cells that have been transfected, either stably or transiently, with a vector capable of expressing one or more polypeptides of interest. In one embodiment, the recombinantly produced antibody is a monoclonal antibody. As used herein, the term “vector” refers to composition of matter which can be used to deliver a nucleic acid of interest to the interior of a cell. Numerous vectors are known including, but not limited to, linear polynucleotides, polynucleotides associated with ionic or amphiphilic compounds, plasmids, and viruses. The term “vector” can include an autonomously replicating plasmid or a virus or a vector or plasmid that is not autonomously replicating. The term “transfection” refers to the introduction of exogenous genetic material into cells to produce genetically modified cells. Vectors can be introduced into a host cell using methods known in the art. For example, a vector can be transferred into a host cell by physical, chemical or biological means. Physical methods for introducing a polynucleotide into a host cell include, but are not limited to, calcium phosphate precipitation, lipofection (including positively charged liposome mediated transfection), particle bombardment, microinjection, DEAE-dextran mediated transfection and electroporation. Biological methods for introducing a vector into a host cell include the use of DNA and RNA vectors, including, for example, viral vectors. Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. In one embodiment, host cells can be genetically engineered to express a recombinant antibody, for example, an antibody of commercial or scientific interest.

The term “cell culture” refers to the growth and propagation of cells outside of a multicellular organism or tissue. Cell culture conditions such as pH, temperature, humidity, atmosphere and agitation can be varied to improve growth and/or productivity characteristics of the cell culture. Host cells may be cultured in suspension or while attached to a solid substrate. Host cells can be cultured in small scale cultures, for example, in a laboratory setting at volumes as low as 25 ml and up to about 50 ml, up to about 100 ml, up to about 150 ml or up to about 200 ml. Alternatively, the cultures can be large scale, for example, at volumes from about 300 ml, 500 ml or 1000 ml and up to about 5000 ml, up to about 10,000 ml and up to about 15,000 ml. Commercial scale bioreactors can also be used, for example, at volumes of up to about 1,000 L, up to about 5,000 L or up to about 10,000 L or more of media. Large scale production of recombinant antibodies by mammalian cells can include continuous, batch and fed-batch culture systems. Host cells may be cultured, for example, in fluidized bed bioreactors, hollow fiber bioreactors, roller bottles, shake flasks, or stirred tank bioreactors, with or without microcarriers, and operated in a batch, fed batch, continuous, semi-continuous, or perfusion mode. Large scale cell cultures are typically maintained for days, or even weeks, while the cells produce the desired protein product(s). In one embodiment, the cell culture conditions results in antibody titers of at least about 1 g/L, 2 g/L, 3 g/L, 4 g/L or 5 g/L and up to about 6 g/L, 7 g/L, 8 g/L, 9 g/L, 10 g/L, 15 g/L or 20 g/L.

Suitable host cells for production of recombinant antibodies include both prokaryotic and eukaryotic cells. Examples of eukaryotic cells include mammalian cells. Examples of mammalian cells suitable for production of recombinant antibodies include, but are not limited to, Chinese hamster ovary (CHO) cells, mouse myeloma (NS0), human embryonic kidney (HEK 293), baby hamster kidney (BHK) cells, Vero cells, HeLa cells, Madin-Darby Canine Kidney (MDCK) cells, CV1 monkey kidney cells, 3T3 cells, myeloma cell lines such as NS0 and NS1, PC12, WI38 cells, COS cells (including COS-1 and COS-7), and C127. In general, mammalian cell cultures are maintained at a pH between about 6.5 and about 7.5 and at a temperature between about 36° C. and about 33° C., typically at about 37° C. and a relative humidity between about 80% and about 95%. Mammalian cell culture media typically contain buffering systems that require a carbon dioxide (CO₂) atmosphere between about 1% and about 10%, or between about 5% and about 6%.

The host cells can be maintained in a variety of cell culture media. The terms “cell culture media” and “cell culture medium” refers to a nutrient solution in which the host cells are grown. Cell culture media formulations are well known in the art. Typically, cell culture media include buffers, salts, carbohydrates, amino acids, vitamins and trace essential elements. The cell culture medium may or may not contain serum, peptone, and/or proteins. Cell culture media may be supplemented with additional or increased concentrations of components such as amino acids, salts, sugars, vitamins, hormones, growth factors, buffers, antibiotics, lipids, trace elements and the like, depending on the requirements of the cells to be cultured and/or the desired cell culture parameters. Various culture media, including serum-free and defined culture media, are commercially available, and include, but are not limited to, Minimal Essential Medium (MEM, Sigma, St. Louis, Mo.); Ham's F10 Medium (Sigma); Dulbecco's Modified Eagles Medium (DMEM, Sigma); Basal Medium Eagle (BME); RPMI-1640 Medium (Sigma); HyClone cell culture medium (HyClone, Logan, Utah); and chemically-defined (CD) media, which are formulated for particular cell types, e.g., CD-CHO Medium (Invitrogen, Carlsbad, Calif.). Supplementary components or ingredients can be added to commercially available media, if desired.

The term “recombinant antibody” as used herein refers to a genetically engineered antibody produced by a cultured host cell. As used herein, the term “heterologous” refers to a recombinant antibody that is produced by a host cell that does not normally express that antibody. It should be noted, however, that a heterologous antibody can include antibodies that are native to an organism, but that have been intentionally altered in some manner. For example, a heterologous antibody can include an antibody that is expressed by a host cell that has been transfected with a vector that expresses the antibody.

The recombinantly produced antibody may be oligoclonal, polyclonal, monoclonal, chimeric, camelised, CDR-grafted, multi-specific, bi-specific, catalytic, humanized, fully human, anti-idiotypic and includes antibodies that can be labeled in soluble or bound form as well as desired fragments, including fragments that exhibit a desired biological activity such as epitope-binding fragments, variants or derivatives thereof, either alone or in combination with other amino acid sequences. The term antibody also includes desired antigen-binding fragments, including, but not limited to Fv, Fab, Fab′, F(ab′)₂ single stranded antibody (svFC), dimeric variable region (Diabody) and disulphide-linked variable region (dsFv). Immunoglobulin molecules can be of any type (e.g., IgG, IgE, IgM, IgD, IgA and IgY), class (e.g., IgG1, IgG2, IgG3, IgG4, IgA1 and IgA2) or subclass. An antibody may be from any species. In one embodiment, the target antibody or desired antigen-binding fragment thereof may be fused to a heterologous polypeptide sequence, such as an affinity tag, to facilitate purification. Examples of affinity tags include, but are not limited to, polyhistidine tags, GFP tags, FLAG tags, GST tags, V5 tags and Myc tags.

In one embodiment, the recombinantly produced antibody is an IgG antibody or a desired antigen-binding fragment thereof. In a more particular embodiment, the recombinantly produced antibody is an anti-type I IFN-α receptor (IFN-αR) monoclonal antibody. In one embodiment, the recombinantly produced antibody is a fully human immunoglobulin G (IgG)-1κ mAb directed against subunit 1 of the IFN-αR1. In a more particular embodiment, the antibody is MEDI-546 or a desired antigen-binding fragment thereof. The term “MEDI-546” refers to an Fc-modified version of the anti-IFNAR 9D4 antibody described in U.S. Pat. No. 7,662,381. The sequence of MEDI-546 is described in U.S. 2011-0059078. MEDI-546 includes a combination of three mutations: L234F, L235E, and P331S, wherein the numbering is according to the EU index as set forth in Kabat, introduced into the lower hinge and CH2 domain of human IgG1, which cause a decrease in their binding to human FcγRI (CD64), FcγRIIA (CD32A), FcγRIII (CD16) and C1 q. See, e.g., US 2011/0059078 and Oganesyan et al. Acta Crystallographica D 64:700-704 (2008), which are hereby incorporated by reference in their entireties. The VH and Vk sequences of MEDI-546 are shown in TABLE 1.

TABLE 1

| | |
|---|---|
| MEDI-546 VH | EVQLVQSGAEVKKPGESLKISCKGSGYIFTNYW |
| (SEQ ID NO: 1) | IAWVRQMPGKCLESMGIIYPGDSDIRYSPSFQG |
| | QVTISADKSITTAYLQWSSLKASDTAMYYCARH |
| | DIEGFDYWGRGTLVTVSS |
| MEDI-546 VK | EIVLTQSPGTLSLSPGERATLSCRASQSVSSSF |
| (SEQ ID NO: 2) | FAWYQQKPGQAPRLLIYGASSRATGIPDRLSGS |
| | GSGTDFTLTITRLEPEDFAVYYCQQYDSSAITF |
| | GQGTRLEIK |

In another embodiment, the target antibody or desired antigen-binding fragment thereof has a light chain variable amino acid sequence of MEDI-546 (SEQ ID NO:2). In another embodiment, the target antibody or desired antigen-binding fragment thereof has a heavy chain variable amino acid sequence of MEDI-546 (SEQ ID NO:1). In another embodiment, the target antibody or desired antigen-binding fragment thereof has a light chain variable amino acid sequence of MEDI-546 (SEQ ID NO:2) and a heavy chain variable amino acid sequence of MEDI-546 (SEQ ID NO:1).

In one embodiment, the antibody includes a heavy chain amino acid sequence having one or more complementarity determining regions (CDRs) of MEDI-546. The terms CDR region or CDR, refer to the hypervariable regions of the heavy and light chains of the immunoglobulin as defined by Kabat et al. (Kabat, E. A. et al. (1991) Sequences of Proteins of Immunological Interest, 5th Edition. US Department of Health and Human Services, Public Service, NIH, Washington or later editions) or Chothia and Lesk (J. Mol. Biol., 196:901-917 (1987)). An antibody typically contains 3 heavy chain CDRs and 3 light chain CDRs. The term CDR or CDRs is used here in order to indicate, according to the case, one of these regions or several, or even the whole, of these regions which contain the majority of the amino acid residues responsible for the binding by affinity of the antibody for the antigen or the epitope which it recognizes. Among the six short CDR sequences, the third CDR of the heavy chain (HCDR3) has a greater size variability (greater diversity essentially due to the mechanisms of arrangement of the genes which give rise to it). It may be as short as 2 amino acids and up to 26 amino acids in length. CDR length may also vary according to the length that can be accommodated by the particular underlying framework. Functionally, HCDR3 plays a role in part in the determination of the specificity of the antibody. One of skill in the art is able to determine CDR regions of an antibody. In general, HCDR1 is about 5 amino acids long, corresponding to Kabat residues 31-35; HCDR2 is about 17 amino acids long, corresponding to Kabat residues 50-65; HCDR3 is about 11 or 12 amino acids long, corresponding to Kabat residues 95-102; LCDR1 is about 11 amino acids long, corresponding to Kabat residues 24-34; LCDR2 is about 7 amino acids long, corresponding to Kabat residues 50-56; and LCDR3 is about 8 or 9 amino acids long, corresponding to Kabat residues 89-97.

The appropriate amino acid residues that encompass the CDRs as defined by each of the above cited references are set forth below in TABLE 2 as a comparison. The exact residue numbers that encompass a particular CDR will vary depending on the sequence and size of the CDR. Those skilled in the art can routinely determine which residues make up a particular CDR given the variable region amino acid sequence of the antibody.

TABLE 2
CDR Definitions¹

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

¹Numbering of all CDR definitions in Table 1 is according to the numbering conventions set forth by Kabat et al.

In one embodiment, the target antibody or desired antigen-binding fragment thereof includes a light chain amino acid sequence that includes one or more light chain CDR sequences for MEDI-546 selected from LCDR1, LCDR2, LCDR3 of the light chain amino acid sequence shown in SEQ ID NO:2, and combinations thereof. In one embodiment, the target antibody or desired antigen-binding fragment thereof includes a heavy chain amino acid sequence that includes one or more of the heavy chain CDR sequences for MEDI-546 selected from HCDR1, HCDR2, HCDR3 of the heavy chain amino acid sequence shown in SEQ ID NO:1, and combinations thereof. In one embodiment, the target antibody or desired antigen-binding fragment thereof includes a light chain amino acid sequence that includes LCDR1, LCDR2, and LCDR3 of the light chain amino acid sequence of MEDI-546 shown in SEQ ID NO: 2 and a heavy chain amino acid sequence that includes HCDR1, HCDR2 and HCDR3 of the heavy chain amino acid sequence of MEDI-546 shown in SEQ ID NO:1.

### 4.4 Antibody Fragmentation Product Impurities

Undesirable antibody fragmentation product impurities can form as a result of indiscriminate enzymatic and non-enzymatic processes. In general, the overall fragmentation pattern observed for a monoclonal antibody is influenced by both structural and solvent conditions. For example, non-enzymatic fragmentation can be influenced by factors such as amino acid sequence, flexibility of the local structure, solvent conditions (e.g., pH and temperature) and the presence of metals or radicals. Non-limiting examples of non-enzymatic fragmentation include direct hydrolysis and beta-elimination, which often result in hinge region fragmentation. In addition to primary structure, the secondary, tertiary and quaternary structures can affect fragmentation patterns due to local flexibility, accessibility to solvent or the proximity of side chains that are remote in sequence. Often, undesirable fragmentation rates are reduced in a pH between about 5 and about 6. A detailed discussion of antibody fragmentation mechanisms can be found in Vlasak and Ionescu, (2011) MAbs, 3(3):253-263.

A variety of antibody fragmentation product impurities are known and can include heavy chain and/or light chain immunoglobulin fragments. Examples of antibody fragment product impurities include, but are not limited to: large hinge (LH), small hinge (SH), heavy-heavy-light (HHL), heavy-light (HL), light-light (LL), heavy-heavy (HH), heavy (H), and light (L). In one embodiment, the antibody fragmentation product impurities includes peptide cleavage fragments, for example, fragmentation products shown in FIG. 1A, including, but not limited to, heavy chain fragmentation products, hinge region fragmentation products, light chain fragmentation products, and combinations thereof.

The term “heavy chain fragmentation product impurity” refers to antibody fragmentation products in which one or more peptide bonds along the length of one or more heavy chains of the antibody have been randomly or unintentionally disrupted. Disruption of one or more peptide bonds can be due to enzymatic or non-enzymatic reactions. In one embodiment, the disruption in the peptide bond in the heavy chain fragmentation product is a N-terminal fragmentation that results in the cleavage of one or more HCDR, for example, one or more of HCDR1, HCDR2 or HCDR3, from the N-terminus of one or more of the heavy chains. In one embodiment shown in FIG. 1A, a heavy chain N-terminal fragment (HC N-frag) is cleaved from a C-terminal fragment, which includes the remainder of the C-terminus of the heavy chain fragment (HC C-fragment), optionally linked to another heavy chain (HC) and one or more light chains (LC) (i.e., HC-LC-LC-HC C-frag). In one embodiment, the disruption in the peptide bond in the heavy chain fragmentation product is a C-terminal fragmentation that results in the cleavage of some or all of the Fc portion from the antibody heavy chain. In one embodiment, a heavy chain C-terminal fragment (HC C-f rag) is cleaved from and N-terminal fragment, which includes the remainder of the N-terminus of the heavy chain (HC N-fragment) optionally linked to another heavy chain (HC) and one or more light chains (LC) (i.e., HC-LC-LC-HC N-frag).

The term “light chain fragmentation product impurity” refers to antibody fragmentation products in which one or more peptide bonds along the length of one or more light chains of the antibody have been randomly or unintentionally disrupted. Disruption of one or more peptide bonds can be due to enzymatic or non-enzymatic reactions. In one embodiment, the disruption in the peptide bond in the light chain fragmentation product is a N-terminal fragmentation that results in the cleavage of one or more LCDR, for example, one or more of LCDR1, LCDR2 or LCDR3, from the N-terminus of one or more of the light chains. In one embodiment, the disruption in the peptide bond in the heavy chain fragmentation product is a C-terminal fragmentation that results in the cleavage of some or all of the Fc portion from the antibody heavy chain. In one embodiment shown in FIG. 1A, one or more light chain fragments (LC) are cleaved from the remainder of the antibody, which can include includes the remainder of the light chain, optionally linked to one or more heavy chains (HC) and one or more light chains (LC) (i.e., HC-HC-LC-Cys).

The term “hinge region fragmentation product impurity” refers to antibody fragmentation products in which one or more peptide bonds along the length of one or more light chains of the antibody have been randomly or unintentionally disrupted near the hinge region of the antibody. Disruption of one or more peptide bonds can be due to enzymatic or non-enzymatic reactions. In one embodiment, the “hinge region fragmentation product” results in the cleavage of a N-terminal fragment that includes a heavy chain variable region linked to a light chain variable region (SHR fragment) from the remainder of the antibody, which includes the C-terminal fragment of the heavy chain, optionally linked to another heavy chain and light chain (LHR Frag).

In another embodiment, antibody fragmentation product impurities includes antibody fragments formed by antibody reduction, for example, by disulfide bond cleavages and include, for example, the fragments shown in FIG. 1B, including, but not limited to fragments that include two heavy chains and a light chain (with a MW of approximately 125 kDa), denoted heavy-heavy-light (HHL), two heavy chains (with a MW of approximately 100 kDa), denoted heavy-heavy (HH), one heavy chain and one light chain (with a MW of approximately 75 kDa), denoted heavy-light (HL), a single heavy chain (with a MW of approximately 50 kDa), denoted heavy (H), a single light chain (with a MW of approximately 25 kDa), denoted light (L) and combinations thereof.

The presence of antibody fragmentation product impurities tends to increase as the upstream titer increases. However, removal of antibody fragmentation product impurities tends to decrease yield. Described herein is a purification process suitable for use with cell culture products having titers of at least about 1 g/L, 2 g/L, 3 g/L, 4 g/L, 5 g/L, 6 g/L, 7 g/L, 8 g/L, 9 g/L and 10 g/L and up to about 5 g/L, 6 g/L, 7 g/L, 8 g/L, 9 g/L, 10 g/L, 15 g/L or 20 g/L, while maintaining a yield of at least about 70%, 75%, 80%, 85%, 90%, 91%, 92%, 93%, 94%, 95%, 96%, 97%, 98% or 99% for at least one step in the purification process. As used herein, the term “step in the purification process” refers to a step such as capture 102, polishing, including a first 104 or a second 105 polishing step, viral inactivation 103, viral filtration 106, and UF/DF 107. In one embodiment, a yield of at least about 70%, 75%, 80%, 85%, 90%, 91%, 92%, 93%, 94%, 95%, 96%, 97%, 98% or 99% is maintained for each step in the purification process. In one embodiment, the purified composition, for example, one or more eluent fractions obtained from the mixed mode chromatography column, includes between about 95% and about 99.9%, or between about 97% and about 99.9%, or between about 98% and 99.9% target antibody or desired antigen-binding fragment thereof, for example, at least about 95%, 96%, 97%, 97.1%, 97.2%, 97.3%, 97.4%, 97.5%, 97.6%, 97.7%, 97.8%, 97.9%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8% or 99.9% target antibody or desired antigen-binding fragment thereof and less than about 3%, 2.9%, 2.8%, 2.7%, 2.6%, 2.5%, 2.4%, 2.3%, 2.2%, 2.1%, 2%, 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3%, 0.2% or 0.1% antibody fragmentation product impurity and less than about 3%, 2.9%, 2.8%, 2.7%, 2.6%, 2.5%, 2.4%, 2.3%, 2.2%, 2.1%, 2%, 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3%, 0.2% or 0.1% aggregate. In one embodiment, the purity of the target in the purified composition is increased at least between about 1% and about 10%, or between about 1% and about 3%, or at least about 0.5%, 1%, 1.5%, 2%, 2.5%, 3%, 3.5%, 4%, 4.5% or 5% and up to about 5.5%, 6%, 6.5%, 7%, 7.5%, 8%, 8.5%, 9%, 9.5% or 10% as compared to purity of the target in the starting material.

Methods for detecting the presence of antibody fragmentation product impurities and determining purity are known and include, for example, size-exclusion chromatography (SEC), for example, high performance size exclusion chromatography (HPSEC), sodium dodecyl sulfate-polyacrylamide gel electrophoresis (SDS-PAGE) and capillary electrophoresis with SDS (CE-SDS) and reverse phase high performance liquid chromatography (RP-HPLC). In one embodiment, the cleavage site can be identified using mass spectrometry (MS) or N-terminal sequencing.

### 4.5 Purification

The first step in the recovery of a recombinantly produced polypeptide, such as an antibody (also referred to herein as a “target polypeptide,” “target antibody” or “target”) from a cell culture is the removal of intact host cells and host cell debris from the culture media, referred to as “harvesting,” to yield a clarified cell culture supernatant that contains the target polypeptide or desired antigen-binding fragment thereof along with other remaining impurities. Harvesting is generally accomplished by centrifugation, flocculation/precipitation, depth filtration and sterile filtration, although other approaches can be used.

Recombinantly produced antibodies can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. When the antibody is secreted into the medium, supernatants from the expression system can be concentrated, for example, using a commercially available protein concentration filter, such as an Amicon or Millipore Pellicon ultrafiltration unit. A protease inhibitor or protease inhibitor cocktail that includes one or more protease inhibitor such as bestatin, aprotinin, pepstatin, leupeptin, 4-(2-Aminoethyl) benzenesulfonyl fluoride hydrochloride (AEBSF), pr phenylmethanesulfonylfluoride (PMSF) may be included to inhibit proteolysis. In other embodiments, one or more antibiotics may be included to prevent the growth of adventitious contaminants. Examples of suitable antibiotics include, but are not limited to, actinomycin D, ampicillin, carbenicillin, cefotaxime, fosmidomycin, gentamicin, kanamycin, neomycin, penicillin, polymyxin B, and streptomycin.

After the clarified cell culture supernatant has been obtained, the target antibody can be further purified by removal of other impurities in the cell culture supernatant that may include, but are not limited to, host cell proteins (HCP), DNA, adventitious and endogenous viruses, endotoxin, aggregates and antibody fragmentation product impurities.

Most purification methods involve some form of chromatography in which target molecules in solution (mobile phase) are separated based on a difference in chemical or physical interaction with a stationary material (solid phase). General chromatographic methods and their use are known to persons skilled in the art. See for example, Sambrook, J., et al. (eds.), Molecular Cloning: A Laboratory Manual, Second Edition, Cold Spring Harbor Laboratory Press, Cold Spring Harbor, N.Y., 1989. Many different methods for recombinant antibody purification are known, and include, but are not limited to affinity chromatography, ion exchange chromatography, hydrophobic interaction chromatography, hydroxylapatite chromatography, size exclusion chromatography, mixed mode chromatography, gel electrophoresis, dialysis and combinations thereof. Other techniques for antibody purification such as fractionation on an ion-exchange column, ethanol precipitation, isoeletric focusing, Reverse Phase HPLC, chromatography on silica, chromatography on heparin, SEPHAROSE™ chromatography on an anion or cation exchange resin (such as a polyaspartic acid column), chromatofocusing, SDS-PAGE, and ammonium sulfate precipitation can also be included in the purification process. Often, a combination of different purification processes are used, such that the different processes separate the recombinantly produced antibody based on different principles, such as affinity, charge, degree of hydrophobicity, and/or size. Many platform processes include a combination of chromatography steps such as anion exchange, cation exchange, hydrophobic interaction and mixed mode resins. Many different chromatography resins are available for each technique, such that a purification scheme can be tailored to the particular recombinant polypeptide. Both automated and gravity flow systems can be coupled to automatic fraction collecting systems.

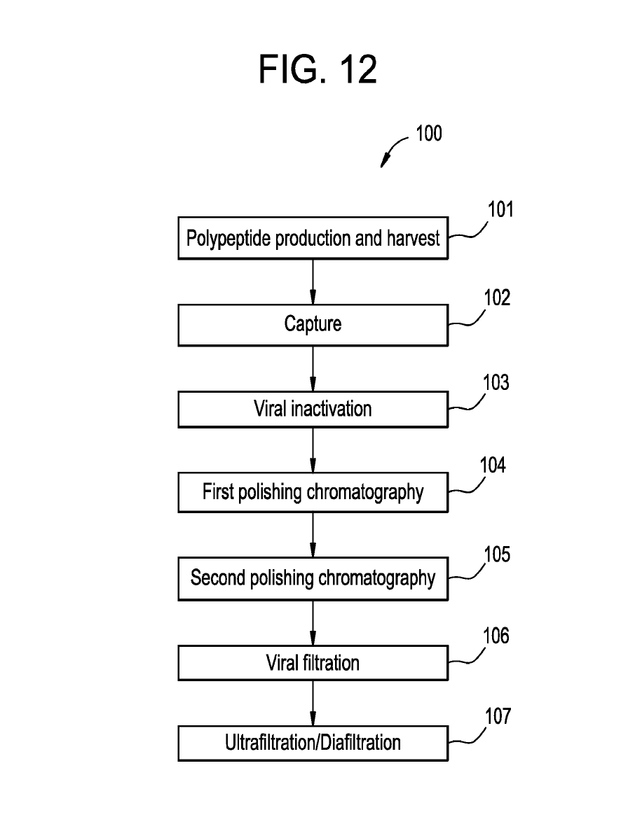

In one embodiment, a combination of purification processes is employed as a purification scheme. One example of purification scheme 100 is shown as a flow chart in FIG. 12. The sample purification scheme 100 includes a first step in which the target antibody or desired antigen-binding fragment thereof is produced, for example, by expression in a host cell and harvested 101. Methods for recombinant antibody production and harvesting are discussed above. The target antibody is then captured 102, for example, using affinity chromatography. In one embodiment, Protein A affinity chromatography is used as a capture step 102 and can effectively remove HCP, DNA and viral contaminants from the process stream. The purification process can also include one or more polishing chromatography steps 104, 105, typically to further decrease DNA, HCP, viral contaminants and to remove aggregate. To improve viral clearance, a viral inactivation step 103 and/or a viral filtration step 106 may also be included in the purification scheme 100. Lastly, the purified product can be concentrated and diafiltered into a final formulation buffer 107. It is noted that the scheme provided in FIG. 12 is merely an example, and variations, for example, in the order of steps, number of steps, and purification methods used for each step, are well within the abilities of one of skill in the art.

In one embodiment, capture 102 is accomplished by affinity chromatography. Affinity chromatography refers to a chromatographic method in which a biomolecule such as a recombinantly produced antibody is separated based on a specific reversible interaction between the polypeptide and a binding partner covalently coupled to the solid phase. Examples of affinity interactions include, but are not limited to the reversible interaction between an antigen and antibody, enzyme and substrate, or receptor and ligand. In one embodiment, affinity chromatography involves the use of microbial proteins, such as Protein A or Protein G. Protein A is a bacterial cell wall protein that binds to mammalian IgGs primarily through their Fc region. Protein A resin is useful for affinity purification and isolation of a variety antibody isotypes, particularly IgG₁, IgG₂, and IgG₄. There are many Protein A resins available that are suitable for use in the purification process described herein. The resins are generally classified based on their backbone composition and include, for example, glass or silica-based resins; agarose-based resins; and organic polymer based resins.

In one embodiment, Protein A affinity chromatography is used to capture a target antibody or desired fragment thereof. The flow rate through an affinity chromatography support is an important parameter for optimizing separation. Although a reduced separation time may be desirable, a flow rate that is too fast may cause the mobile phase to move past the solid phase faster than the diffusion time necessary to reach the internal bead volume. Generally, a flow rate of at least about 50 cm/h, 100 cm/h, 150 cm/h, 200 cm/hour or 250 cm/hour and up to about 300 cm/hour, 350 cm/hour, 400 cm/hour, 450 cm/hour or 500 cm/hr is used. The column dimensions can also be varied. While laboratory bench scale columns generally have a column diameter of less than 1 cm, or less than 5 cm, large scale or commercial production scales can use columns having diameters of up to 1 meter or even up to 2 meters. For large scale or commercial production, the column bed height is generally at least about 10 cm, 15 cm or 20 cm, and up to about 25 cm or 30 cm.

The composition of the buffer solutions and the volume of buffer solutions used in connection with Protein A purification can be varied. The term “buffer” or “buffered solution” refers to a solution that is able to resist changes in pH. Often a buffer is made of a weak conjugate acid-base pair, for example, a weak acid and its conjugate base or a weak base and its conjugate acid. In some buffers, the buffering agent is supplied as a crystalline acid or base, for example, Tris is supplied as a crystalline base, which is dissolved in water to form a buffering solution. The pH of the buffering solution can be adjusted using an appropriate acid or base. For example, hydrochloric acid (HCl) can be used to adjust the pH of a Tris buffering solution. Other buffers are prepared by mixing two components, such as a free acid or base and a corresponding salt, in ratios that achieve the desired pH. For example, a sodium citrate buffer solution can be made and adjusted to the desired pH by combining citric acid and trisodium citrate to form a solution with the desired pH. Other buffers are made by mixing a buffer component and its conjugate acid or base. For example, a phosphate buffer can be made by mixing monobasic and dibasic sodium phosphate solutions in a ratio to achieve a desired pH. In another embodiment, a sodium bicarbonate buffer system can be prepared by combining solutions of sodium carbonate and sodium bicarbonate to form a buffer solution having a desired pH.

In one embodiment, the column is equilibrated with an “equilibration buffer” prior to loading. The term “equilibration buffer” refers to a buffer that can be used to remove undesired residual from the column matrix and to prepare the solid phase of the column matrix for loading the target protein, for example, by adjusting the pH of the column. When used for antibody purification, the pH of the equilibration buffer is at least about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, or 7.9, and up to about 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0. In one embodiment, the equilibration buffer includes a buffering agent such as tris(hydroxymethyl)aminomethane (often referred to as “Tris”) (pH range 5.8-8.0), 4-(2-hydroxyethyl)-1-piperazineethanesulfonic acid (HEPES) (pH range 6.8-8.2), 3-(N-morpholino)propanesulfonic acid (MOPS) (pH range 6.5-7.9) or other phosphate buffering agents (pH 5.8-8.0) at a concentration of at least about 10 mM, 25 mM, 50 mM or 75 mM and up to about 100 mM, 125 mM or 150 mM. In one embodiment, the pH of the buffering solution can be adjusted using an appropriate acid or base, such as hydrochloric acid (HCl) or sodium hydroxide/potassium hydroxide (NaOH/KOH). In one embodiment, the equilibration buffer includes at least about 10 mM, 15 mM, or 20 mM and up to about 25 mM, 30 mM, 50 mM or 100 mM sodium phosphate at a pH of at least about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, or 7.9, and up to about 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0. Additionally, the buffer may include one or more additives to increase protein purity, stability, and function, including, but not limited to reducing agents such as 2-mercaptoethanol (BME), dithiothreiotol (DDT) or Tris(2-carboxyethyl)phosphine (TCEP) to protect against oxidative damage, protease inhibitors, including but not limited to leupeptin, pepstatin A and phenylmethanesulfonylfluoride (PMSF) to inhibit endogenous proteases from degrading the target polypeptide, metal chelators, including but not limited to ethylenediaminetetraacetic acid (EDTA) and ethylene glycol tetraacetic acid (EGTA), to inactivate metalloproteases, osmolytes, including but not limited to glycerol, detergents and sugars to stabilize protein structure or ionic stabilizers, including but not limited to salts such as NaCl, KCl and (NH₄)₂SO₄ to enhance solubility. In one embodiment, the column is equilibrated using at least about 5, and up to about 10 or 20 column volumes of the equilibration buffer prior to loading the recombinantly produced polypeptide onto the column.

In one embodiment, a clarified cell culture supernatant is loaded onto the column. In one embodiment, the clarified cell culture supernatant is loaded onto the column after the column has been equilibrated with an equilibration buffer. In a further embodiment, the clarified cell culture supernatant is loaded onto the column in combination with a loading buffer. The term “loading buffer” refers to a buffer that is combined with a composition that includes the target polypeptide prior to loading the target onto a column. In general, the target polypeptide is loaded at a concentration of at least about 1 mg/ml, 5 mg/ml, 10 mg/ml, 15 mg/ml, 20 mg/ml or 25 mg/ml and up to about 30 mg/ml, 35 mg/ml, 40 mg/ml, 45 mg/ml, 50 mg/ml, 75 mg/ml or 100 mg/ml. In one embodiment, clarified cell culture supernatant is diluted with a loading buffer at a ratio of about 1:1, 1:2 or 1:3, for example, to achieve a desired concentration for the target polypeptide and/or to adjust the pH of the solution. In other embodiments, the clarified cell culture supernatant is loaded directly onto the column (i.e., the supernatant is not diluted with a loading buffer). In one embodiment, the column is re-equilibrated with an equilibration buffer after the clarified cell culture supernatant has been loaded. In a more particular embodiment, the column is re-equilibrated with at least about 5 and up to about 10 or 20 column volumes of the equilibration buffer or loading buffer after the target polypeptide is loaded onto the column. In general, the target polypeptide is loaded onto the Protein A column at a pH of at least about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, or 7.9, and up to about 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0. In some embodiments, the loading buffer is the same as the equilibration buffer. In other embodiments, the loading buffer and the equilibration buffer are not the same. In other embodiments, the loading buffer is also used as a wash buffer to wash the column after loading.

In one embodiment, the loading buffer includes a buffering agent such as histidine, tris(hydroxymethyl)aminomethane (often referred to as “Tris”) (pH range 5.8-8.0), 4-(2-hydroxyethyl)-1-piperazineethanesulfonic acid (HEPES) (pH range 6.8-8.2), 3-(N-morpholino)propanesulfonic acid (MOPS) (pH range 6.5-7.9) or other phosphate buffering agents, such as sodium phosphate or phosphate-citrate buffers (pH 5.8-8.0) at a concentration of at least about 10 mM, 20 mM, 30 mM, 40 mM or 50 mM and up to about 50 mM, 60 mM, 70 mM, 80 mM, 90 mM, or 100 mM. In one embodiment, the pH of the buffering solution can be adjusted using an appropriate acid or base, such as hydrochloric acid (HCl) or sodium hydroxide/potassium hydroxide (NaOH/KOH). When used for antibody purification, the pH of the loading buffer is generally adjusted to at least about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, or 7.9, and up to about 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0. In a more particular embodiment, the loading buffer includes at least about 10 mM, 15 mM or 20 mM and up to about 25 mM, 30 mM, 50 mM or 100 mM sodium phosphate at has a pH of at least about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, or 7.9, and up to about 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0. In one embodiment, the column is re-equilibrated after loading using at least about 5, and up to about 10 or 20 column volumes of the equilibration or loading buffer. Additionally, the equilibration buffer may include one or more additives to increase protein purity, stability, and function, including, but not limited to reducing agents such as 2-mercaptoethanol (BME), dithiothreiotol (DDT) or Tris(2-carboxyethyl)phosphine (TCEP) to protect against oxidative damage, protease inhibitors, including but not limited to leupeptin, pepstatin A and phenylmethanesulfonylfluoride (PMSF) to inhibit endogenous proteases from degrading the target polypeptide, metal chelators, including but not limited to ethylenediaminetetraacetic acid (EDTA) and ethylene glycol tetraacetic acid (EGTA), to inactivate metalloproteases, osmolytes, including but not limited to glycerol, detergents and sugars to stabilize protein structure or ionic stabilizers, including but not limited to salts such as NaCl, KCl and (NH₄)₂SO₄ to enhance solubility.

The term “wash buffer” refers to a buffer that is passed over the column material after the target composition has been loaded onto the column and prior to elution of the recombinantly produced target polypeptide. The wash buffer may serve to remove one or more contaminants, for example, antibody fragmentation product impurities, from the column material, without substantial elution of the target. In general, the wash buffer has a pH of at least about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, or 7.9, and up to about 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0. In one embodiment, the process includes one wash buffer, wherein the column is washed using at least about 5, or up to about 10 or 20 column volumes of a single wash buffer. In other embodiments, the process may include more than one wash buffer, for example, the process may include two different wash buffers. For example, the process may include a first wash step in which the column is washed using at least about 5, or up to about 10 or 20 column volumes of a first wash buffer and a second wash step in which the column is washed using at least about 5, or up to about 10 or 20 column volumes of a second wash buffer. In one embodiment, at least one wash buffer is the same as the equilibrating buffer. In another embodiment, at least one wash buffer is different from the equilibration buffer.

Additionally, the wash buffer may include one or more additives to increase protein purity, stability, and function, including, but not limited to reducing agents such as 2-mercaptoethanol (BME), dithiothreiotol (DDT) or Tris(2-carboxyethyl)phosphine (TCEP) to protect against oxidative damage, protease inhibitors, including but not limited to leupeptin, pepstatin A and phenylmethanesulfonylfluoride (PMSF) to inhibit endogenous proteases from degrading the target polypeptide, metal chelators, including but not limited to ethylenediaminetetraacetic acid (EDTA) and ethylene glycol tetraacetic acid (EGTA), to inactivate metalloproteases, osmolytes, including but not limited to glycerol, detergents and sugars to stabilize protein structure or ionic stabilizers, including but not limited to salts such as NaCl, KCl and (NH₄)₂SO₄ to enhance solubility.

The term “elution buffer” refers to a buffer used to elute (i.e., remove) the target polypeptide from the column. The elution pH can vary depending upon the binding affinity of the polypeptide to the column. Some antibodies demonstrate a higher binding affinity and may require a lower elution pH. In general, the pH of the elution buffer is lower than the pH of the loading buffer. Typically, the elution buffer has a pH of at least about 2.0, 2.1, 2.2, 2.3, 2.4, 2.5, 2.6, 2.7, 2.8, 2.9, 3.0, 3.1, 3.2, 3.3, 3.4, or 3.5 and up to about 3.6, 3.7, 3.8, 3.9, 4.0, 4.1, 4.2, 4.3, 4.4, 4.5, 4.5, 4.7, 4.8, 4.9 or 5.0. Examples of elution buffers include buffers including sodium citrate, citric acid or acetic acid at a concentration of at least about 25 mM, 50 mM and up to about 100 mM, 150 mM or 200 mM. In one embodiment, the elution buffer includes at least about 25 mM, 50 mM and up to about 100 mM, 150 mM or 200 mM sodium citrate at has a pH of at least about 2.0, 2.1, 2.2, 2.3, 2.4, 2.5, 2.6, 2.7, 2.8, 2.9, 3.0, 3.1, 3.2, 3.3, 3.4, or 3.5 and up to about 3.6, 3.7, 3.8, 3.9, 4.0, 4.1, 4.2, 4.3, 4.4, 4.5, 4.5, 4.7, 4.8, 4.9 or 5.0. Additionally, the elution buffer may include one or more additives to increase protein purity, stability, and function, including, but not limited to reducing agents such as 2-mercaptoethanol (BME), dithiothreiotol (DDT) or Tris(2-carboxyethyl)phosphine (TCEP) to protect against oxidative damage, protease inhibitors, including but not limited to leupeptin, pepstatin A and phenylmethanesulfonylfluoride (PMSF) to inhibit endogenous proteases from degrading the target polypeptide, metal chelators, including but not limited to ethylenediaminetetraacetic acid (EDTA) and ethylene glycol tetraacetic acid (EGTA), to inactivate metalloproteases, osmolytes, including but not limited to glycerol, detergents and sugars to stabilize protein structure or ionic stabilizers, including but not limited to salts such as NaCl, KCl and (NH₄)₂SO₄ to enhance solubility. In one embodiment, the target molecule is eluted using at least 5 and up to 10 or up to 20 column volumes of elution buffer. The eluate can be monitored using techniques well known to those skilled in the art, for example by monitoring the absorbance using a spectrophotometer set at OD₂₈₀ nm. In one embodiment, the Protein A purification step has a recovery rate of at least about 70%, 75%, 80%, 81%, 82%, 83%, 84% or 85% and up to about 86%, 87%, 88%, 89%, 90% or 95%. Recovery can be determined, for example, by calculating the percentage of protein in the eluate relative to the amount that was loaded onto the column.

Polishing chromatography steps 104, 105 provide additional viral, host cell protein (HCP), endotoxin and DNA clearance, as well as assist in the removal of aggregates, unwanted product variants and other minor contaminants. Polishing steps 104, 105 generally include one or more chromatographic steps such as ion exchange chromatography (anion exchange and/or cation exchange), mixed mode chromatography, hydrophobic interaction chromatography, and combinations thereof.

In one embodiment, the purification process includes at least one ion exchange chromatography step. The term “ion exchange chromatography” refers to a chromatographic process using an immobile matrix that carries covalently bound charged substituents. The “ion exchange material” has the ability to exchange its counter ions, which are not covalently bound, for similarly charged binding partners or ions in the surrounding solution. Polypeptides have numerous functional groups that can have either positive or negative charges. Ion exchange chromatography separates polypeptides based on net charge, which is dependent on the pH and/or ionic concentration of the mobile phase. Polypeptides can thus be separated by adjusting the pH and/or ionic concentration of the mobile phase. In some embodiments, the target polypeptide is captured by the column and then eluted (also referred to as “bind and elute” mode). In other embodiments, the target polypeptide flows through the column and contaminants are bound (also referred to as a “flow through mode”). Elution from an ion exchange material is generally achieved by increasing the ionic strength of the buffer to compete with the target for charged sites of the ion exchange matrix. The elution process be gradual (gradient elution) or stepwise (step elution) and the eluate can be monitored, for example, using a UV spectrophotometer set at OD₂₈₀ nm.

Depending on the charge of the counter ions, “ion exchange chromatography” can be referred to as “cation exchange,” “anion exchange,” or “mixed-mode ion exchange.” The term “cation exchange” refers to a chromatographic method having a solid phase that is negatively charged with free cations available for exchange with cations in an aqueous solution passed over or through the column. Cation exchange chromatography can be used to purify a recombinant antibody if the target is maintained under conditions in which the target is positively charged. For example, the solution can be titrated so that the solution pH is lower than the isoelectric point of the target. Other positively charged impurities may also be bound to the cationic column resin in addition to the target. As such, the target can be recovered by elution from the column under conditions (e.g., pH and salt concentration) in which the target elutes while impurities remain bound to the resin. Cation exchange resins can include strong acidic ligands such as sulphopropyl, sulfoethyl and sulfoisobutyl groups or weak acidic ligand such as carboxyl groups. Examples of commonly used cation exchange resins include carboxymethyl (CM), sulfoethyl(SE), sulfopropyl(SP), phosphate (P) and sulfonate(S) resins.

The term “anion exchange” refers to a chromatographic method having a solid phase that is positively charged with free anions available for exchange with anions in an aqueous solution passed over or through the solid phase. Anion exchange columns are typically operated in a flow through mode, such that negatively charged impurities are bound to the resin while the positively charged target is recovered in the flow through stream. However, anion exchange columns may also be used in a bind and elute mode, depending upon the pl of the target and the impurities to be removed. Examples of positively charged groups that are used in anion exchange include weakly basic groups such as diethylamino ethyl (DEAE) or dimethylamino ethyl (DMAE) and strongly basic groups such as quaternary amine (Q) groups, trimethylammonium ethyl (TMAE) or quaternary aminoethyl (QAE).

In one embodiment, the eluate obtained from the Protein A capture step 102 is subjected to one, more than one, or two ion exchange separation steps in which the second ion exchange separation involves a separation based on the opposite charge than the first ion exchange separation. For example, if an anion exchange step 104 is employed after capture 102, the second ion exchange chromatographic step 105 may be a cation exchange step. Conversely, if capture 102 was followed by a cation exchange step 104, that step would be followed by an anion exchange step 105. Alternately, in other embodiments the purification scheme 100 may include only a cationic exchange step or only an anionic exchange step.

In another embodiment, the purification scheme can include mixed mode chromatography as a capture step and/or as a polishing step. Mixed mode chromatography involves the use of solid phase chromatographic supports that employ a combination of chemical mechanisms, including, but not limited to, ionic interactions (i.e., cation and anion), hydrogen bonds, and/or hydrophobic interactions to absorb proteins or other solutes. Examples include chromatographic supports that exploit combinations of two or more of the following mechanisms: anion exchange (AEX), cation exchange (CEX), hydrophobic interaction (HIC), hydrophilic interaction, hydrogen bonding, pi-pi bonding and metal affinity. In one embodiment, the purification scheme includes a mixed mode anion exchange (AEX) and hydrophobic interaction (HIC) chromatography step. In another embodiment, the purification scheme includes a mixed mode cation exchange (CEX) and hydrophobic interaction (HIC) chromatography step. In a more particular embodiment, the purification scheme includes a Capto Adhere™ mixed mode (AEX and HIC) chromatography step. Multiple mechanisms (ionic bonding, H-bonding, and hydrophobic bonding) are combined and responsible for the chromatographic behavior of protein on Capto Adhere™. In another embodiment, the purification scheme includes a Capto MMC™ mixed mode (CEX and HIC) chromatography step. Although mixed mode chromatography, such as Capto Adhere™, is known to be effective at capturing antibodies and reducing host cell proteins during capture (Pezzini et al. (2011), J. Chromatogr A, 1218:8197); for the removal of aggregate during monoclonal antibody purification (Gao et al. (2013) J. Chromatogr A, 1294:70) and for viral clearance (Connell-Crowley et al. (2013) Biotechnol Bioeng, 110:1984), until now, mixed mode chromatography has not been used to separate monoclonal antibody fragmentation product impurities from a target monoclonal antibody. However, the inventors have found that mixed mode chromatograpy is a robust and effective method for the removal of antibody fragmentation product impurities that can easily be integrated into a platform manufacturing process.

In one embodiment, the purification scheme includes a Capto Adhere™ purification step. Loading conditions, such as sample load, conductivity and pH can be varied to favor yield and/or to favor clearance of impurities. In one embodiment, the mixed mode purification step can be performed in a bind and elute mode, in which the target is adsorbed to the column matrix and contaminants pass through the column. In another embodiment, the mixed mode purification step can be performed in a flow through mode, in which the target passes through the column, while contaminants are adsorbed to the column matrix.

In general, a starting material that includes the target antibody or desired antibody fragment thereof and a loading buffer is loaded onto the column. In one embodiment, the sample load is between about 10 g/L and about 500 g/L, more typically between about 25 g/L and 300 g/L. When used in a bind and elute mode, the sample load is generally at least about 1 g/L, 5 g/L, 10 g/L, 15 g/L, 20 g/L, 25 g/L or 30 g/L and up to about 30 g/L, 35 g/L, 40 g/L, 45 g/L or 50 g/L. When used in a flow through mode, the sample load is generally between about 25 g/L and 500 g/L or 75 g/L and 250 g/L, for example, at least about 25 g/L, 50 g/L, 75 g/L, 100 g/L, 125 g/L, 150 g/L, 175 g/L or 200 g/L and up to about 100 g/L, 150 g/L, 175 g/L, 200 g/L, 225 g/L, 250 g/L, 275 g/L, 300 g/L, 350 g/L, 400 g/L or 500 g/L. In general, purification of a starting material with a lower load capacity (for example, less than about 250 g/L, 200 g/L, 150 g/L, 100 g/L, 75 g/L, 50 g/L or 25 g/L) results in a product with increased purity of the target antibody or desired antigen-binding fragment thereof. In one embodiment, the conductivity of the starting material is between about 0 mS/cm and 30 mS/cm, for example, at least about 1 mS/cm 2 mS/cm, 3 mS/cm, 4 mS/cm, 5 mS/cm, 6 mS/cm, 7 mS/cm, 8 mS/cm, 9 mS/cm or 10 mS/cm and up to about 5 mS/cm, 6 mS/cm, 7 mS/cm, 8 mS/cm, 9 mS/cm, 10 mS/cm, 15 mS/cm, 20 mS/cm, 25 mS/cm or 30 mS/cm. In general, increased purity of the target antibody or desired antigen-binding fragment thereof is observed when the starting material has a lower conductivity, for example, a conductivity of less than about 10 mS/cm, 9 mS/cm, 8 mS/cm, 7 mS/cm, 6 mS/cm, 5 mS/cm, 4 mS/cm, 3 mS/cm, 2 mS/cm or 1 mS/cm. In one embodiment, the starting material that is loaded onto the mixed mode column includes between about 85% and about 99%, or between about 90% and about 98%, or between about 97% and 98% target antibody or desired antigen-binding fragment thereof, for example, at least about 88%, 89%, 90%, 91%, 92%, 93%, 94%, 95%, 96%, 97%, 97.1%, 97.2%, 97.3%, 97.4%, 97.5%, 97.6%, 97.7%, 97.8%, 97.9%, 98%, 98.1%, 98.2%, 98.3%, 98.4%, 98.5%, 98.6%, 98.7%, 98.8%, 98.9%, 99% target antibody or desired antigen-binding fragment thereof and up to about 97%, 97.1%, 97.2%, 97.3%, 97.4%, 97.5%, 97.6%, 97.7%, 97.8%, 97.9%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8% or 99.9%, and between about 1% and about 10% antibody fragmentation product impurity, or between about 1% and about 3% antibody fragmentation product impurity, for example, at least about 1%, 1.5%, 2%, 2.5%, 3%, 3.5%, 4%, 4.5%, 6%, 6.5%, 7%, 7.5%, 8%, 8.5%, 9%, 9.5%, or 10% and up to about 10% or 15% antibody fragmentation product impurity, and between about 1% and about 5% aggregate, or between about 1% and about 3% aggregate, for example, less than about 3%, 2.9%, 2.8%, 2.7%, 2.6%, 2.5%, 2.4%, 2.3%, 2.2%, 2.1%, 2%, 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3%, 0.2% or 0.1% aggregate. Methods for determining the residual levels of antibody fragments are known and include, for example RPLC and HPSEC.

Suitable buffer systems for use with mixed mode resins can vary depending upon many factors, including, but not limited to, the desired pH range and whether the column is used in a bind and elute mode, or in a flow through mode. Common buffers used in column chromatography include, but are not limited to phosphate buffers, for example, sodium phosphate or potassium phosphate; citrate buffers, including, but not limited to sodium citrate; sodium acetate; and biological buffers, including but not limited to, 3-(N-morpholino)propanesulfonic acid (MOPS), (4-(2-hydroxyethyl)-1-piperazineethanesulfonic acid (HEPES), tris(hydroxymethyl)aminomethane (TRIS); and combinations thereof.

In general, loading buffers are used at a concentration of between about 1 mM and about 40 mM, for example, at least about 1 mM, 5 mM, 10 mM, 15 mM, 20 mM or 20 mM and up to about 10 mM, 15 mM, 20 mM, 25 mM, 30 mM, 35 mM or 40 mM. In one embodiment, the pH of the loading buffer is between about 4 and about 9, between about 5 and about 8, or between about 5.5 and about 6.5, or between about 5.7 and about 6.1, or at least about 4, 4.5, 5, 5.5, 6, 6.5 or 7 and up to about 6, 6.5, 7, 7.5, 8, 8.5 or 9. In general, purification at a higher pH (for example, a pH of at least about 5, or at least about 5.5 and up to about 6, or up to about 6.5) tends to increase the removal of antibody fragmentation product impurities and target purity as compared to lower pH (for example, a pH of less than about 5). In one embodiment, the conductivity of the loading buffer is less than about 10 mS/cm, 9 mS/cm, 8 mS/cm, 7 mS/cm, 6 mS/cm, 5 mS/cm, 4 mS/cm, 3 mS/cm, 2 mS/cm or 1 mS/cm.

In one embodiment, the purification scheme 100 includes at least one hydrophobic interaction separation as a polishing step 104 or 105. “Hydrophobic interaction chromatography” (HIC) refers to chromatographic separation based on the reversible interaction between a polypeptide and a hydrophobic ligand bound to the solid phase of the chromatography resin. Hydrophobic interaction chromatography is often used to remove protein aggregates, such as antibody aggregates, and process-related impurities. During HIC, the target polypeptide binds to the column at a high salt concentration and is eluted by decreasing the salt concentration. Since the interaction between the target polypeptide and the hydrophobic ligand are enhanced by the use of buffers with high ionic strength, HIC can be a suitable purification step for use after ion echange chromatography. Various ions can be arranged in a so-called soluphobic series depending on whether they promote hydrophobic interactions (salting-out effects) or disrupt the structure of water (chaotropic effect) and lead to the weakening of the hydrophobic interaction.

In other embodiments, the purification scheme can include “hydrophobic charge induction chromatography” (HCIC) as a polishing step 104 or 105. HCIC is based on the pH-dependent behavior of ligands that ionize at low pH. HCIC employs heterocyclic ligands at high densities such that hydrophobic interaction between the target polypeptide and the column material are possible without the need for a high salt concentration. Elution in HCIC can be accomplished by lowering the pH to produce charge repulsion between the ionizable ligand and the bound protein.

In one embodiment, the purification scheme 100 includes one or more viral inactivation 103 and/or viral clearance 106 steps, for example, to remove endogenous retroviruses and adventitious viruses. In one embodiment, a viral inactivation step 103 can be included after the target molecule is captured 102. Viral inactivation techniques are known and include, for example, heat inactivation (pasteurization), pH inactivation, disruption of the lipid envelope using solvent/detergent, UV and γ-ray irradiation and the use of chemical inactivating agents. In one embodiment, viral inactivation includes a step of low pH viral inactivation, which includes incubating the mixture for a period of time at low pH, neutralizing the pH and removing particulates by filtration. In one embodiment, the low pH viral inactivation includes titrating the recombinant antibody to a pH between about 2 and about 5, or between about 3 and about 4, or between about 3.3 and about 3.8. The pH of the sample mixture may be lowered by any suitable acid including, but not limited to, citric acid, acetic acid, caprylic acid, or other suitable acids. The choice of pH level depends on the stability profile of the target and other buffer components. Typically, the titrated solution is incubated for at least about 30 or 45 minutes and up to about 1, 1.5 hours, or 2 hours, typically at room temperature. After viral inactivation, the pH of the recombinant antibody solution can be adjusted to a more neutral pH, for example, between about 4.5 to about 8.5, or between about 4.5 and about 5.5 prior to continuing the purification process.

In another embodiment, a viral clearance step 105, such as a viral filtration, can be included in the purification scheme. Virus-retentive filters are commercially available and include ultrafilters or microfilters such as hydrophilic polyethersulfone (PES), hydrophilic polyvinylidene (PVDF) and regenerated cellulose filters. Based on the size of viruses that are removed, virus filters can be categorized into retrovirus filters and parvovirus filters.

Mixed mode chromatography, such as Capto Adhere™ can be easily integrated into a platform monoclonal antibody purification process. Many platform processes include a protein A capture step followed by a low pH inactivation, polishing steps, such as anion exchange, cation exchange, and combinations thereof and finally nanofiltration and formulation ultrafiltration/diafiltration. In many platform processes, the low pH product obtained after the low pH viral inactivation step is low in conductivity and must be neutralized to approximately pH 7.5 before it can be loaded onto the polishing resin. Use of a mixed mode resin can be advantageous in that the process can be performed at a lower pH (e.g., between pH 5.5-6.0) while maintaining low conductivity, requiring less volumetric addition of base.

In one embodiment, the purification scheme includes an ultrafiltration (UF) and/or diafiltration (DF) step 107 to further purify and concentrate the target. UF/DF can increase the concentration of the target macromolecule as well as replace buffering salts with a particular formulation buffer. Ultrafiltration (UF) refers to a type of membrane filtration in which hydrostatic pressure forces a liquid against a semipermeable membrane. Suspended solids and solutes of high molecular weight, such as the target, are retained in the retentate, while water and low molecular weight solutes pass through the membrane in the filtrate. In this manner, the target is concentrated as liquid and salt are removed. Generally, the low molecular weight composition in the concentrate remains constant so the ionic strength of the concentrated solution remains relatively constant. “Diafiltration” refers to a method that uses ultrafiltration membranes to remove, replace, or lower the concentrations of salts or buffering components from solutions containing proteins, such as antibodies, peptides, nucleic acids, and other biomolecules. Continuous diafiltration (also referred to as constant volume diafiltration) involves washing out the original buffer salts (or other low molecular weight species) in the retentate by adding water or a new buffer, such as a formulation buffer, to the retentate to form a formulation containing the recombinantly produced polypeptide. Typically, the new buffer is added at the same rate as filtrate is being generated such that the retentate volume and product concentration does not change appreciably during diafiltration.

### 4.5. Formulations

In one embodiment, the target polypeptide is prepared in a liquid formulation. In a more particular embodiment, the target polypeptide in the liquid formulation is an antibody or antibody fragment, for example, a recombinantly produced antibody or desired antibody fragment thereof. In one embodiment, the liquid formulation includes an aqueous carrier, such as water. In one embodiment, the liquid formulation is sterile. In another embodiment, the liquid formulation is homogeneous. In another embodiment, the formulation is isotonic. In one embodiment, the liquid formulation includes at least about 1 mg/ml, 5 mg/ml, 10 mg/ml, 20 mg/ml, 25 mg/ml, 50 mg/ml, 75 mg/ml, 100 mg/ml, 125 mg/ml, 150 mg/ml, 175 mg/ml, 200 mg/ml, 250 mg/ml, or 300 mg/ml of the target. In one embodiment, the formulation has a pH of at least about 3.0, 3.5, 4.0, 4.5, 5.0, 5.1, 5.2, 5.3, 5.4, 5.5, 5.6, 5.7, 5.8, 5.9, 6.0, 6.1, 6.2, 6.3, 6.4 or 6.5 and up to about 6.6, 6.7, 6.8, 6.9, 7.0, 7.5, 8.0, 8.5, or 9.0. The formulation may also include common excipients and/or additives, including, but not limited to buffering agents, sugars, saccharides, salts, surfactants, solubilizers, diluents, binders, stabilizers, lipophilic solvents, amino acids, chelators, preservatives or combinations thereof.

In another embodiment, the target is prepared as a lyophilized formulation. In a more particular embodiment, the target in the lyophized formulation is an antibody or antibody fragment, for example, a recombinantly produced antibody or desired fragment thereof. As used herein, the term “lyophilized” refers to a formulation that has been subjected to a drying procedure, such as lyophilization, where at least 50% of moisture has been removed from the starting material. In one embodiment, the lyophilized formulation includes a lyoprotectant. The term “lyoprotectant” refers to a molecule which, when combined with the target, significantly prevents or reduced chemical and/or physical instability of the target upon lyophilization and subsequent storage. Lyoprotectants include, but are not limited to, sugars and their corresponding sugar alcohols; an amino acid such as monosodium glutamate, arginine or histidine; a methylamine such as betaine; a lyotropic salt such as magnesium sulfate; a polyol such as trihydric or higher molecular weight sugar alcohols, e.g. glycerin, dextran, erythritol, glycerol, arabitol, xylitol, sorbitol, and mannitol; propylene glycol; polyethylene glycol; Pluronics®; and combinations thereof. Additional examples of lyoprotectants include, but are not limited to, glycerin and gelatin, and the sugars mellibiose, melezitose, raffinose, mannotriose and stachyose. Examples of reducing sugars include, but are not limited to, glucose, maltose, lactose, maltulose, iso-maltulose and lactulose. Examples of non-reducing sugars include, but are not limited to, non-reducing glycosides of polyhydroxy compounds selected from sugar alcohols and other straight chain polyalcohols. Examples of sugar alcohols include, but are not limited to, monoglycosides, compounds obtained by reduction of disaccharides such as lactose, maltose, lactulose and maltulose. The glycosidic side group can be either glucosidic or galactosidic. Additional examples of sugar alcohols include, but are not limited to, glucitol, maltitol, lactitol and iso-maltulose. In specific embodiments, trehalose or sucrose is used as a lyoprotectant. In one embodiment, the lyoprotectant is added to the formulation in a “lyoprotecting amount” which means that, following lyophilization of the protein in the presence of the lyoprotecting amount of the lyoprotectant, the target essentially retains its physical and chemical stability and integrity upon lyophilization and storage. A “reconstituted” formulation is one which has been prepared by dissolving a lyophilized formulation in a diluent such that the target is dispersed in the reconstituted formulation. The reconstituted formulation is suitable for administration to a patient. The “diluents” includes pharmaceutically acceptable diluents, including, but not limited to, sterile water, bacteriostatic water for injection (BWFI), a pH buffered solution (e.g. phosphate-buffered saline), sterile saline solution, Ringers solution or dextrose solution. In an alternative embodiment, diluents can include aqueous solutions of salts and/or buffers. In one embodiment, the a recombinantly produced antibody or fragment thereof is included in a lyophilized formulation at a concentration of at least about 10 mg/ml, 20 mg/ml, 30 mg/ml, 40 mg/ml or 50 mg/ml and up to about 50 mg/ml, 60 mg/ml, 70 mg/ml, 80 mg/ml, 90 mg/ml or 100 mg/ml. In a more particular embodiment, the lyophilized formulation includes an amino acid, such as histdine, arginine or glutamic acid as a buffer at concentration of at least about 10 mM, 15 mM, 20 mM or 25 mM and up to about 30 mM, 40 mM or 50 mM. In one embodiment, the lyophilized formulation includes a sugar such as trehalose or sucrose at a concentration of at least about 50 mM, 100 mM, 150 mM, 175 mM, 200 mM or 225 mM and up to about 250 mM or 300 mM. In one embodiment, the lyophilized formulation includes at least about 0.01%, 0.02% 0.03%, 0.04% or 0.05% (w/v) and up to about 0.06%, 0.07%, 0.08%, 0.09% or 0.1% (w/v) of a surfactant, such a polysorbate 80. In one embodiment, the lyophilized formulation has a pH of at least about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, or 7.9, and up to about 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0.

In one embodiment, the target is a recombinantly produced antibody or desired antigen-binding fragment thereof that is formulated for parenteral administration. In one embodiment, the formulation is injectable. In one embodiment, the target is formulated for intravenous, subcutaneous, or intramuscular administration.

### 5. EXAMPLES

The following examples are provided to help illustrate the invention described herein. They are not intended to limit or define the scope of this invention.

The reagents employed in the examples are commercially available or can be prepared using commercially available instrumentation, methods, or reagents known in the art. The examples illustrate various aspects of the invention and practice of the methods of the invention. The examples are not intended to provide an exhaustive description of the many different embodiments of the invention. Thus, although the invention has been described in some detail by way of illustration and example for purposes of clarity of understanding, those of ordinary skill in the art will realize readily that many changes and modifications can be made thereto without departing from the spirit or scope of the appended claims.

All publications, patents and patent applications mentioned in this specification are herein incorporated by reference into the specification to the same extent as if each individual publication, patent or patent application was specifically and individually indicated to be incorporated herein by reference.

### Example 1

The cycle 1 process used in the purification of a monoclonal antibody (MEDI-546) was performed using a 2 g/L cell culture product. As shown in Table 3, the purified cycle 1 material was highly pure (i.e., the monomer content was high and fragment levels were low). However, a 4 g/L cycle 2 cell culture product produced a starting material that contained higher fragment levels and lower monomer levels. Described herein is a process that is able to remove fragmented species from a high concentration cell culture product (i.e., 4 g/L) while matching the cycle 1 product quality and maintaining a >90% yield for each operation in the purification process. Analysis of the fragmentation profiles demonstrated that the cycle 1 and cycle 2 cell culture materials were producing identical fragmentation species. However, the cycle 2 material contained increased levels of large and small hinge fragments (LHF/SHF). FIG. 1 shows a schematic drawing of the peptide cleavage sites as well as the RPLC assay profiles for the two cell culture materials.

TABLE 3
Purity of Cycle 1 vs Cycle 2 Products

| | HPSEC | | | RPLC |
|---|---|---|---|---|
| | % | % | % | % |
| Material | Monomer | Aggregate | Fragment | Fragment |
| Purification Targets | >99.0 | <1.0 | | <2.6 |
| Cycle 1 Reference | 99.5 | 0.5 | 0.0 | 2.4 |
| Standard | | | | |
| Cycle 2 Capture Product | 98.4-99.8 | 0.3-0.8 | 0.0-1.4 | 1.9-3.2 |

### Materials and Equipment

MabSelect SuRe™, Capto Q™, Capto Phenyl™, and Capto Adhere™ resins were purchased from GE Healthcare (Uppsala, Sweden). The C0HC depth filters were purchased from EMD Millipore (Billerica, Mass.). Acetic acid, arginine, sodium acetate, sodium acetate trihydrate, sodium chloride, sodium phosphate dibasic, sodium phosphate monobasic, tris base, tris hydrochloride, and urea were purchased from Avantor Performance Chemicals (Center Valley, Pa.). Ethylene glycol was purchased from Sigma-Aldrich (St. Louis, Mo.). For chromatographic experiments, resins were packed in Vantage™ columns purchased from Millipore (Billercia, Mass.). Purification was controlled by unicorn 5.2 software on an Akta Explorer purchased from GE Healthcare (Uppsala, Sweden). For statistical analysis, Jmp version 8.0 software was purchased from SAS (Cary, N.C.).

### Cell Culture Preparation

A cryovial containing a suspension of cells was thawed and the cell suspension was used to inoculate a shake flask containing growth medium. The shake flask was placed in a temperature controlled incubator with a constant level of carbon dioxide and air present and agitated to keep the cells aerated and well mixed. The seed train (i.e., the process in which the total number of cells is increased) was continued by passaging the cells into larger volumes of growth medium. Whereas the early cell expansion steps were completed in shake flasks, the seed train was extended into bioreactors until sufficient biomass was accumulated to inoculate the production bioreactor. The seed bioreactors were kept at constant temperature and agitated and aerated to maintain the cells at a targeted rate of growth. The production bioreactor was allowed to grow under controlled temperature, pH, dissolved oxygen, aeration, and agitation until such a time as a sufficient cell mass was accumulated. Once a set level of cell mass was obtained, the production bioreactor was fed nutrients in bolus additions to enhance cell growth and antibody production. The production bioreactor was fed every other day until day twelve, at which point the bioreactor was chilled and prepared for harvest and purification.

### SEC-HPLC

A test sample was injected onto a TosoHaas G3000SWXL column (7.8 mm×30 cm). The sample was eluted isocratically with 0.1 M disodium phosphate containing 0.1 M sodium sulfate and 0.05% sodium azide, pH 6.8, at a flow rate of 1.0 ml/minute. Eluted protein was detected using UV absorbance at 280 nm. The results were reported as the area percent of the product monomer peak compared to all other peaks excluding the buffer-related peak observed at approximately 12 minutes. Peaks eluting earlier than the monomer peak were recorded as percent aggregate. Peaks eluting after the monomer peak were recorded as percent other.

### RP-HPLC

The sample (10 μg) was injected onto a PLRP-S 8 micron 4000 A column, 2.0×150 mm. Mobile phase A was 0.05% trifluoroacetic acid (TFA) in water and mobile phase B was 0.05% TFA in acetonitrile. The flow rate was 0.75 mL/minute and the column temperature was set to 75° C. The protein was eluted using a gradient of 34-40% TFA in acetonitrile (mobile phase B) from 5-12 minutes and was monitored using UV absorbance at 280 nm. The reported fragment percent was the total area of all the fragments divided by the total peak area of all the fragments and the intact antibody.

### Purification of Monoclonal Antibody/Capto Adhere™ Load

Cell culture harvested product was captured on MabSelect Sure™ resin. After loading, the column was successively washed in high salt and low salt Tris buffer. The antibody was then eluted by a low pH acetate buffer. The eluate underwent viral inactivation by low pH treatment with acetic acid. The viral inactivation product was used as the input material to the Capto Adhere™ studies.

### Capto Adhere™ Range Finding DOE

Studies were performed to determine the impact of pH, conductivity, and load capacity on the Capto Adhere™ product quality and yield. The impact of pH was first explored. Viral inactivated product (HPSEC 99.5% monomer, 0.4% aggregate, 0.0 fragment, RPLC 2.8% fragment) was pH adjusted to increments between 5 and 8. The pH adjusted material was loaded onto the column at 100 g/L capacity in equilibration buffer at the respective pH and the flow through was collected. Acid was then applied to strip the column. The yield and product qualities of the flow through and strips were analyzed.

Once the pH was chosen that resulted in desired yield and purity, the impact of conductivity and load capacity were studied. A jmp full factorial 2³ design was performed using the same starting material at tight pH values around the pH 6 target (5.5-6.5), a wide range of load capacity (75-300 g/L), and a wide range of conductivity (0-30 mS/cm). Flow through and strip fractions were collected and analyzed for yield and purity.

### Capto Adhere™ Robustness/Centerpoint/Scale-Up

A jmp fractional factorial composite design was used to study robustness. Ranges were designed to include worst case fit-to-plant manufacturing values of key parameters. Starting material had worst case purities of 97.7% monomer, 0.6% aggregate, 1.7% fragment by HPSEC and 2.8% fragment by RPLC. Load capacity ranged from 50 to 110 g/L. Load pH ranged from 5.5 to 6.0. Load conductivity ranged from 3 to 7 mS/cm. Load buffer conductivity ranged from 3.56 to 4.39 mS/cm. Purification products were analyzed for purity and yield.

The operational parameters determined from the robustness experiments were integrated into the monoclonal antibody purification process and performed at both bench and pilot scales. The C0HC depth filtered viral inactivation product was adjusted to pH 5.9±0.2 with 1 M acetic acid and the conductivity was monitored to ensure it was <7 mS/cm. The adjusted material was loaded to 100 g/L onto a Capto Adhere™ column that was equilibrated in 50 mM acetate pH 5.9±0.2. The load was chased with equilibration buffer and the flow through was collected. The column was striped by the addition of water for injection (WFI) and then 100 mM acetic acid. Sanitization was performed with 1 N sodium hydroxide prior to storage in 2% benzyl alcohol, 100 mM acetate pH 5.0.

### Canto Adhere™ Fragment Spiking

Capto Adhere™ load was spiked to a purity of 89.7% monomer, 2.7% aggregate, 7.9% fragment by HPSEC and 9.2% fragment by RPLC. The load material was purified to analyze the impact of pH (pH range 5 to 8), conductivity (0 to 30 mS/cm), and load capacity (75 to 250 g/L). Flow through products were analyzed for purity and yield.

### Canto Adhere™ Mechanism

Non-spiked adjusted loads each containing 2.8% fragment by RPLC were purified on Capto Q™ and Capto Phenyl™ resins for comparison to Capto Adhere™. A jmp full factorial DOE was performed on Capto Q™ to study the impact of pH (pH 7-8), conductivity, (0-20 mS/cm), and load capacity (0 to 300 g/L). Studies were also performed on Capto Phenyl™ resins in flow through mode to study the impact of conductivity (5 to 90 mS/cm).

The fragment removal phase modified mechanism experiments were performed according to Hou and Cramer (2011) J. Chromatogr A, 1218:7813. Capto Adhere™ adjusted loads were separately spiked with 20% (v/v) ethylene glycol, 2M urea, and 0.1 M arginine. After incubation for two hours at room temperature, the spiked loads were purified according to the MEDI-546 process. For each experiment, flow through and strip peaks were collected and analyzed for yield and purity.

### Range Finding DOE:

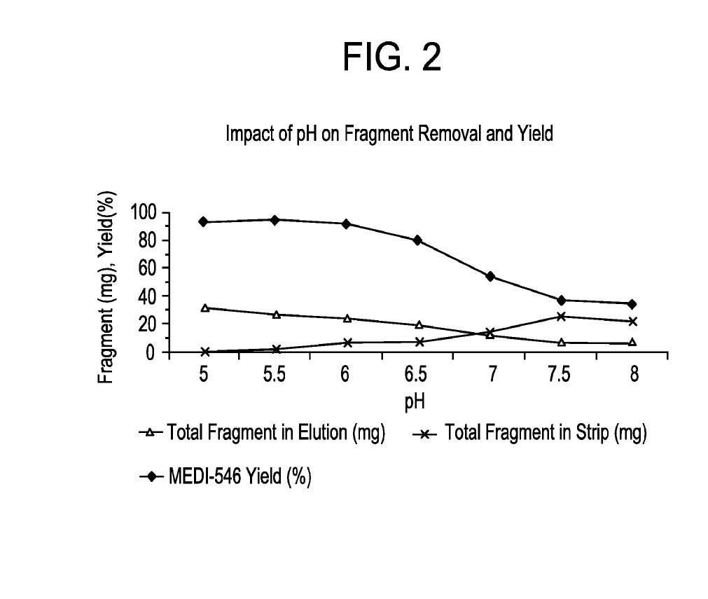

Capto Adhere™ was evaluated for removal of antibody fragmentation product impurities. First, the impact of pH on product quality and yield were studied. FIG. 2 shows the fragment mass balances and yields for each pH studied. Each purification load contained 30 mg of antibody fragmentation product impurities by RPLC. As the pH increased from 5 to 8, increasing amounts of antibody fragmentation product impurities bound to the column and were separated from the flow through material. Although the level of fragment decreased in the flow through product with increasing pH, yield decreased.

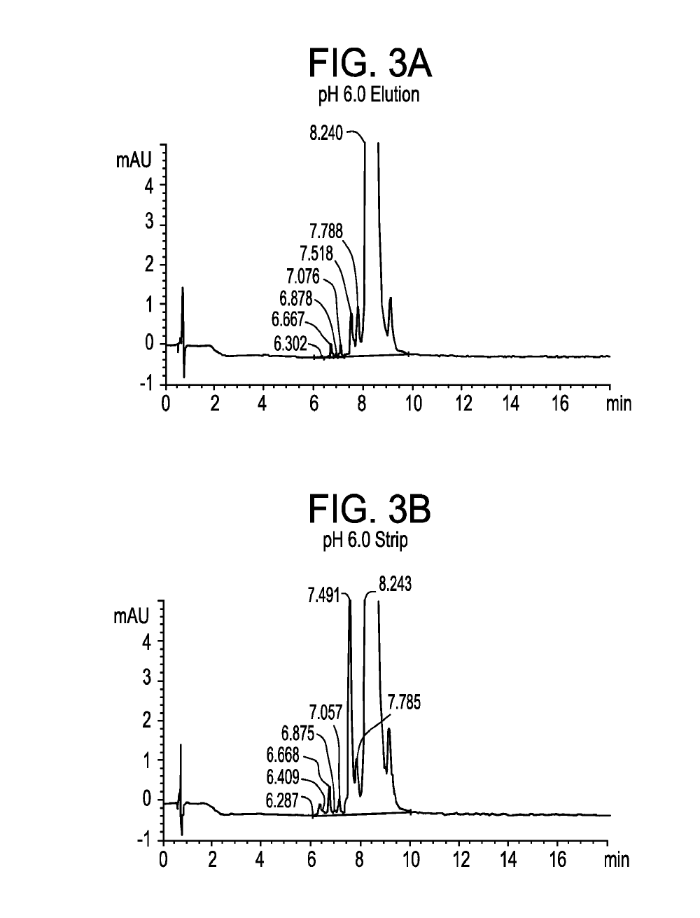

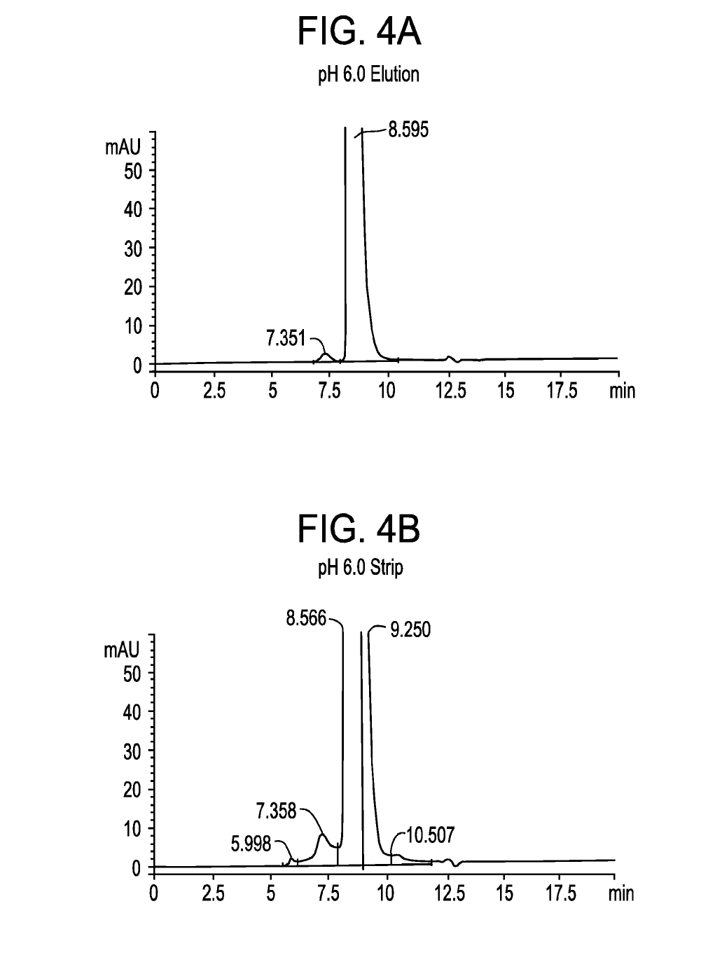

The RPLC profiles showing the fragmentation profiles of the flow through products and low pH strip fractions from each purification were analyzed. Comparison of the profiles showed that LHF/SHF bound to the Capto Adhere™ resin and therefore separated from the flow through products. Examination of corresponding HPSEC profiles shows that the flow through products contained high levels of monomer while the strip products were enriched for antibody fragmentation product impurities. FIGS. 3A and 3B show RPLC profiles for the pH 6 purification for strip and elution, respectively. FIGS. 4A and 4B show RPLC profiles for the pH 6 purification, for strip and elution, respectively. At pH 6, the fragment purity of 2.4482%, monomer purity of 99.7, and yield greater than 90% met the cycle 2 purification target criteria. Table 4 provides the percentages of the various fragments in the elution and strip fractions at pH 6 as determined by RPLC. Table 5 provides the percentages of monomer, aggregate and fragment in the elution and strip fractions at pH 6 as determined by HPSEC.

TABLE 4

| | Elution | Strip |
|---|---|---|
| IgG Minus HC 1-137 | 1.0132% | 1.2099% |
| LHR/SHR Frag | 0.9611% | 6.4549% |
| HC 241-446 | 0.1759% | 0.2159% |
| HC 1-137 | 0.0567% | 0.1031% |
| LC 1-214 | 0.1785% | 0.4174% |
| HC 326-446, 331-446 | 0.0628% | 0.3012% |
| Total | 2.4482% | 8.7024% |

TABLE 5

| | HPSEC | | |
|---|---|---|---|
| | % | % | % |
| | Monomer | Aggregate | Fragment |
| pH 6.0 Elution | 99.7 | 0.3 | 0.0 |
| pH 6.0 Strip | 89.1 | 2.2 | 8.7 |

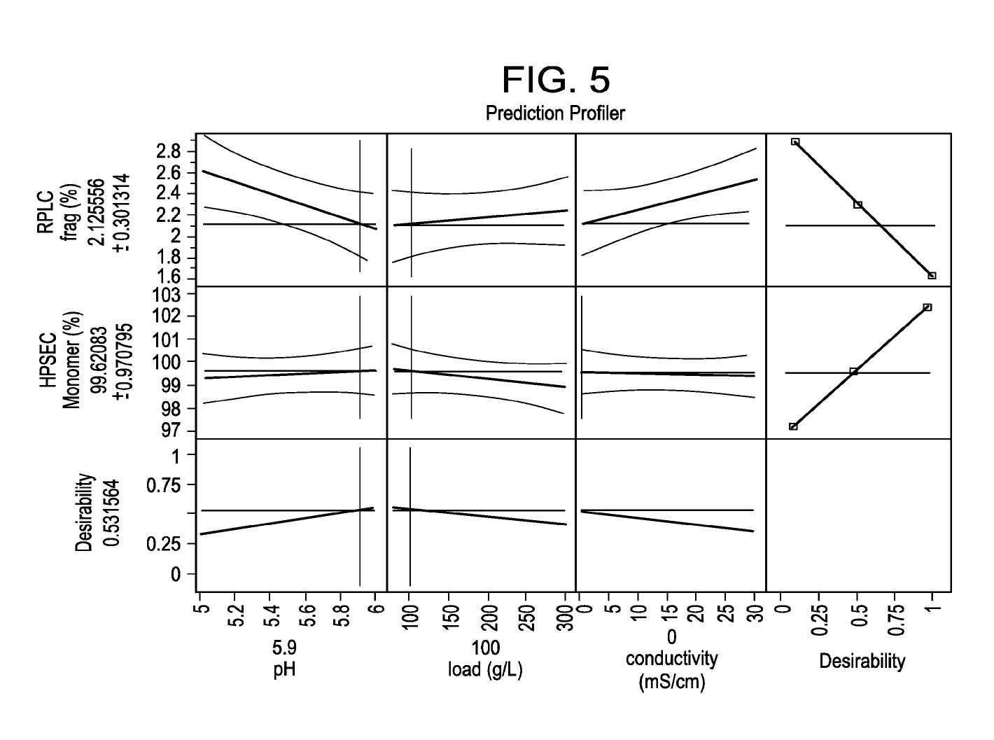

The impact of conductivity and load capacity were next studied at pH ranges surrounding the pH 6 target. The jmp screening design results of the flow through material are shown in FIG. 5. As expected, purification at higher pH resulted in increased removal of antibody fragmentation product impurities and monomer purity when compared to lower pH. Load capacity and conductivity were also key factors for removal of antibody fragmentation product impurities. In general, increased monomer purity was observed at lower load capacity and low conductivity.

### Scale-Up Robustness/Centerpoint/Scale-Up

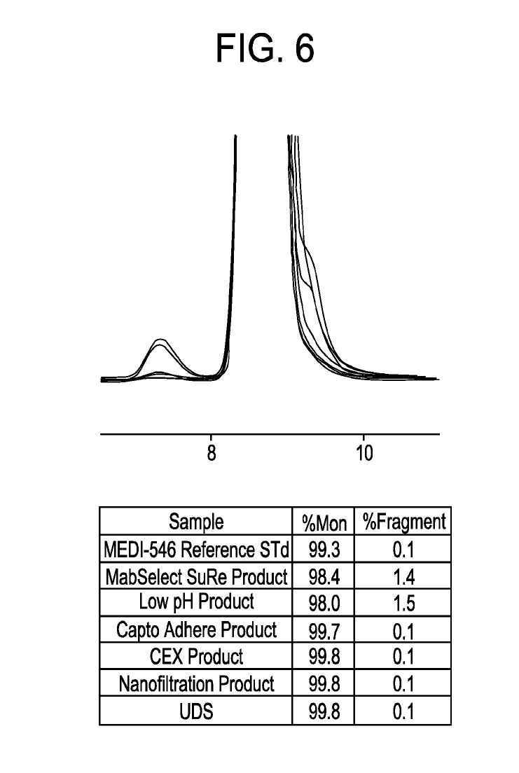

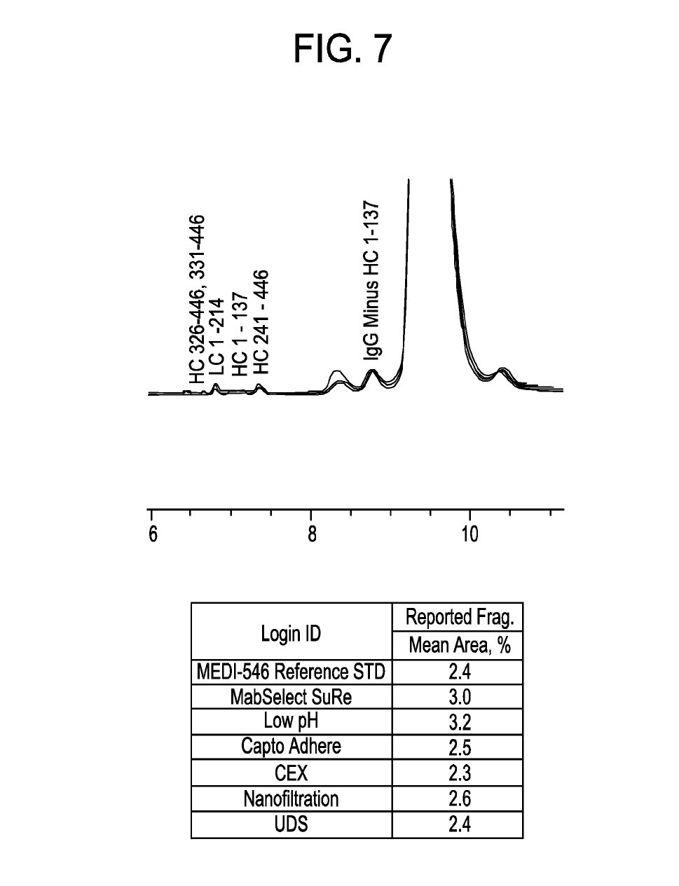

Robustness studies were performed to analyze how the Capto Adhere™ removal of antibody fragmentation product impurities would function when integrated into an industrial monoclonal antibody purification process. Manufacturing facilities require operational space away from the centerpoint parameters so that the product qualities and yields are known and acceptable when produced form any point in the space. A combination of process parameters including load capacity, load pH, load conductivity, and buffer conductivity were studied. Load material for these experiments was 97.7% monomer, 0.6% aggregate, 1.7% fragment by HPSEC and 2.8% fragment by RPLC. FIGS. 6 and 7 show the product quality results by HPSEC and RPLC, respectively. At the worst case manufacturing conditions, capacity of 110 g/L, pH of 5.5, load conductivity of 7 mS/cm, and buffer conductivity of 4.39 mS/cm, HPSEC monomer was 99.4% and RPLC fragment was 2.5%, meeting the cycle 2 targets. The response surface profiler showed that less than 2.0% fragment by RPLC is expected in the product when the load pH is between 5.7 and 6.0 regardless of the other parameters.

The robust centerpoint Capto Adhere™ fragment removal operation was integrated into a platform purification process and successfully performed at pilot scale. FIGS. 6 and 7 show the Capto Adhere™, HPSEC and RPLC chromatograms from each intermediate in the process compared to the cycle 1 reference standard. The cell culture cycle 2 product was captured by MabSelect SuRe™. The intermediate was 98.4% monomer by HPSEC and 3.0% fragment by RPLC, with increased LHF/SHF compared to the cycle 1 material. After low pH viral inactivation, the product showed a slight increase in fragment. Application of the low pH viral inactivation product to Capto Adhere™ chromatography resulted in an in increase in purity to acceptable levels of 99.6% HPSEC monomer and 2.5% RPLC fragment. The fragment remained at a lower level in the cation exchange, nanofiltration, and formulated products. The final purified material met the cycle 2 targets and matched the cycle 1 material.

### Fragment Reduction by Canto Adhere™

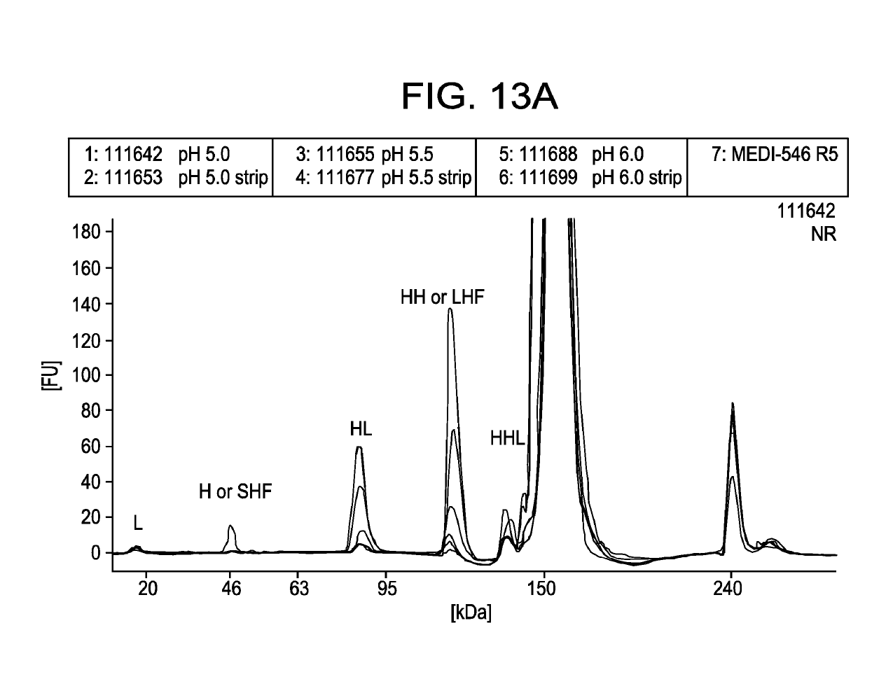

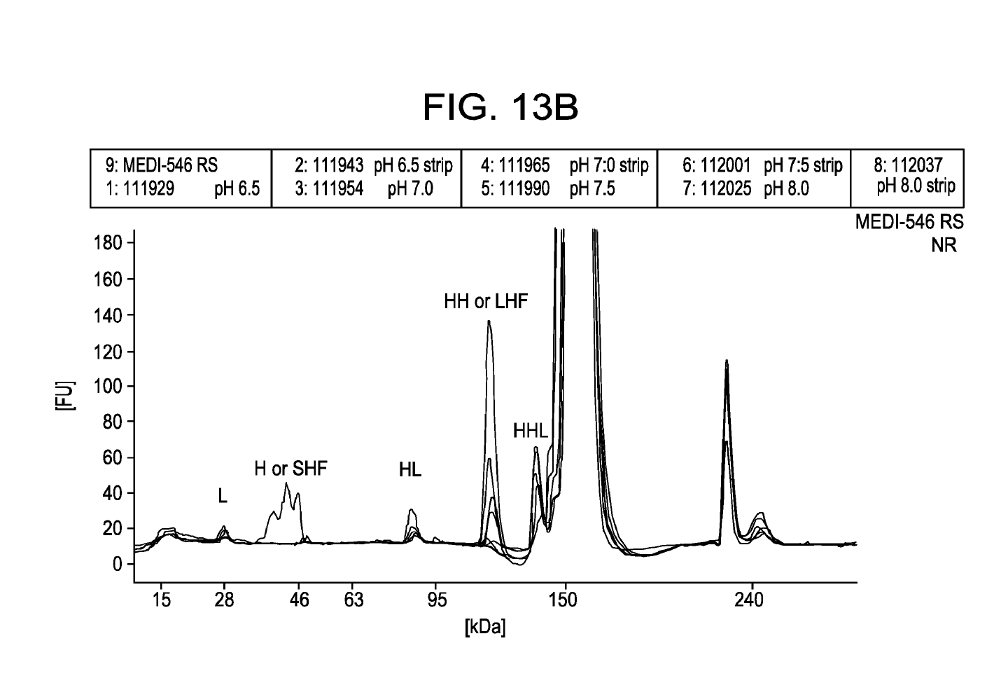

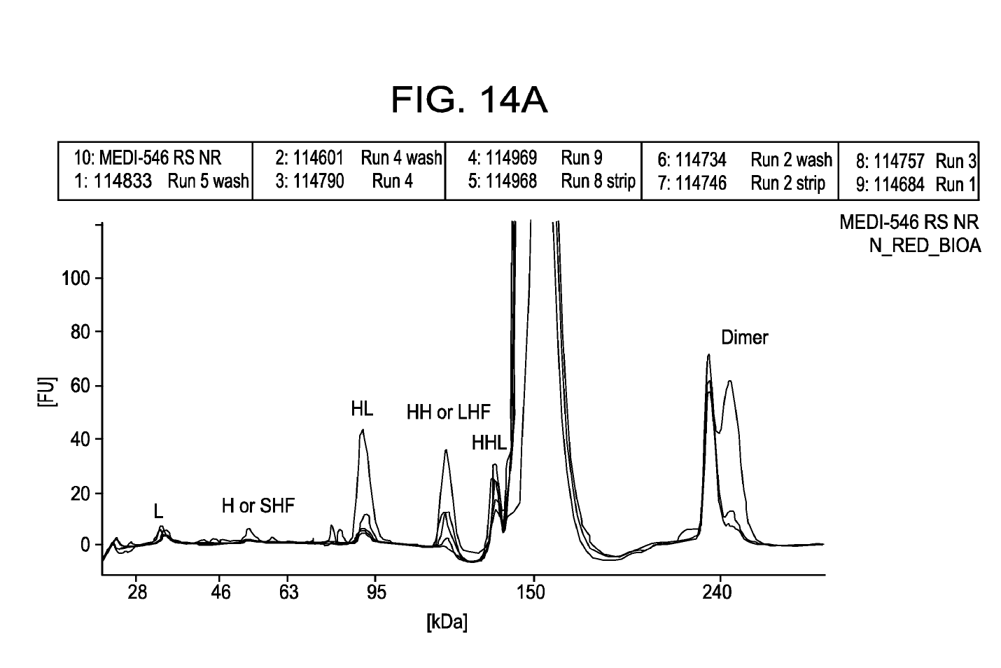

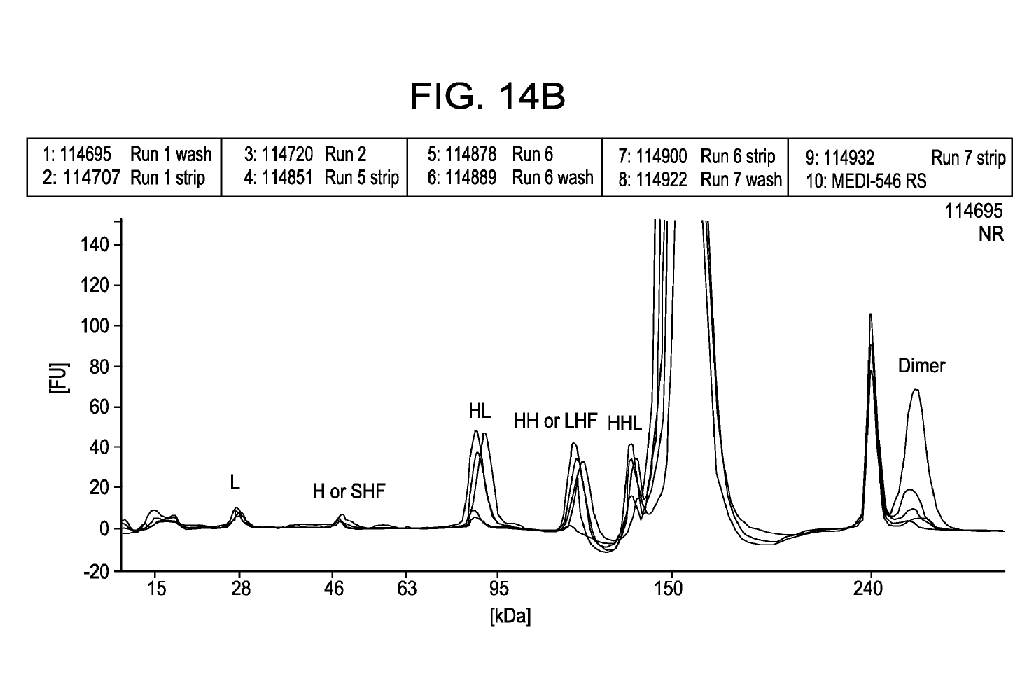

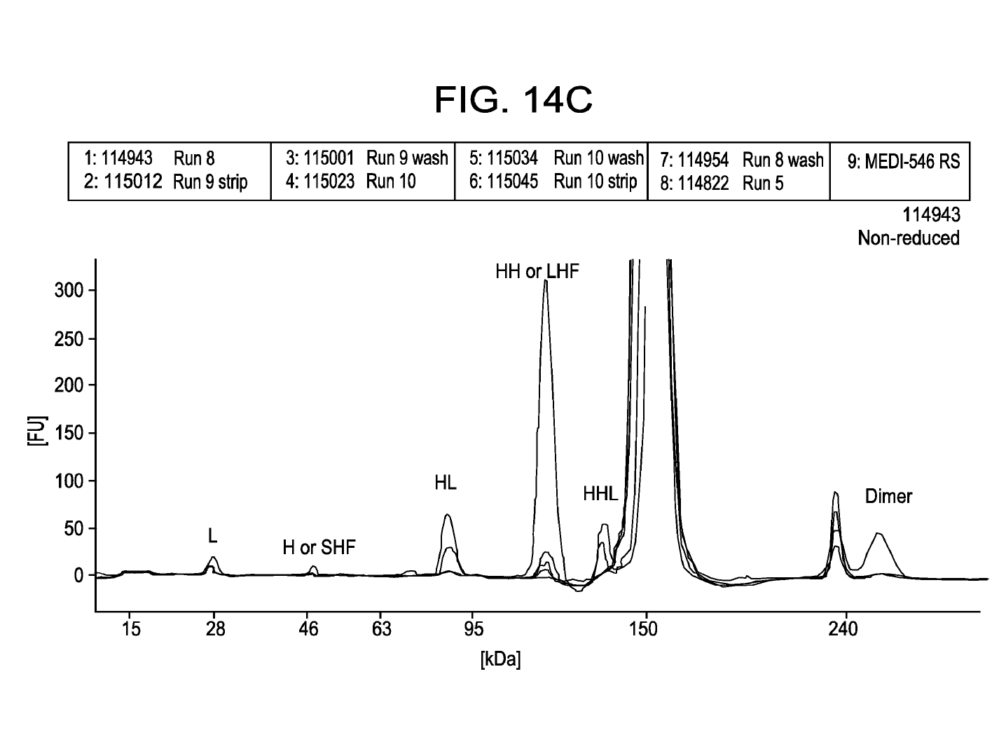

FIGS. 13 and 14 demonstrate a decrease in levels of HHL, HH, HL, H and L during purification using Capto Adhere™ (as described above) as determined using a Bioanalyzer assay. FIG. 13 contains Bioanalyzer chromatograms showing levels of antibody reduction fragments including HHL, HH or LHF, HL, H or SHF and L fragments during purification using Capto Adhere™ resin at pH 5, 5.5, 6, 6.5, 7, 7.5 and 8. The elution profiles are comparable to the reference standard whereas the strip profiles are enriched for reduction fragments. The data shows that reduction fragments bind to Capto Adhere™ across a broad range of pH indicating that the resin can be utilized to purify monoclonal antibodies from their reduced forms. FIG. 14 is a Bioanalyzer chromatogram showing levels of HHL, HH or LHF, HL, H or SHF and L fragments, as well as dimer resulting from purifications using Capto Adhere™ resin Columns were loaded to between 75 and 300 g/L with material was that was adjusted between pH 5 to 6, and conductivity between 0 to 30 mS/cm. The results show that reduced antibody can bind to Capto Adhere™ and allowing for purification of intact monoclonal antibody across a range of load capacity, pH and conductivity.

### Spiking-Power

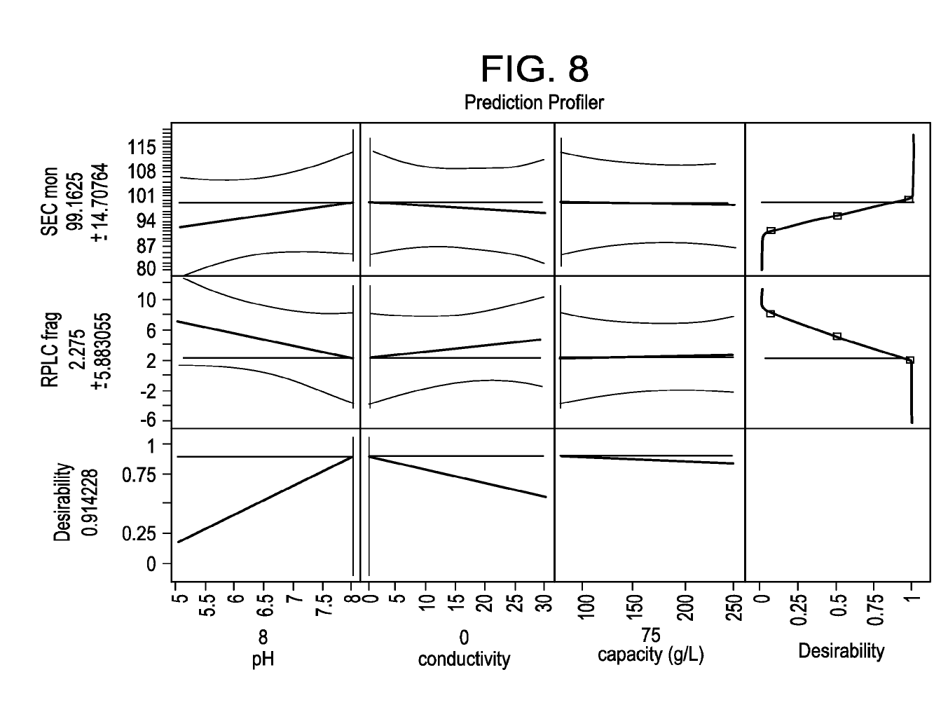

After successful integration into the manufacturing process, efforts were made to determine the overall fragment separation capability of Capto Adhere™. FIG. 8 shows the results of the spiking experiments. Although yield would be impacted, load material that was spiked to 7.9% HPSEC fragment and 9.2% RPLC fragment was able to be purified to cycle 2 targets when the purification is performed at pH 8 with low load capacity and conductivity. The results also show that the Capto Adhere™ column can remove up to 2.0% RPLC fragment.

### Mechanism

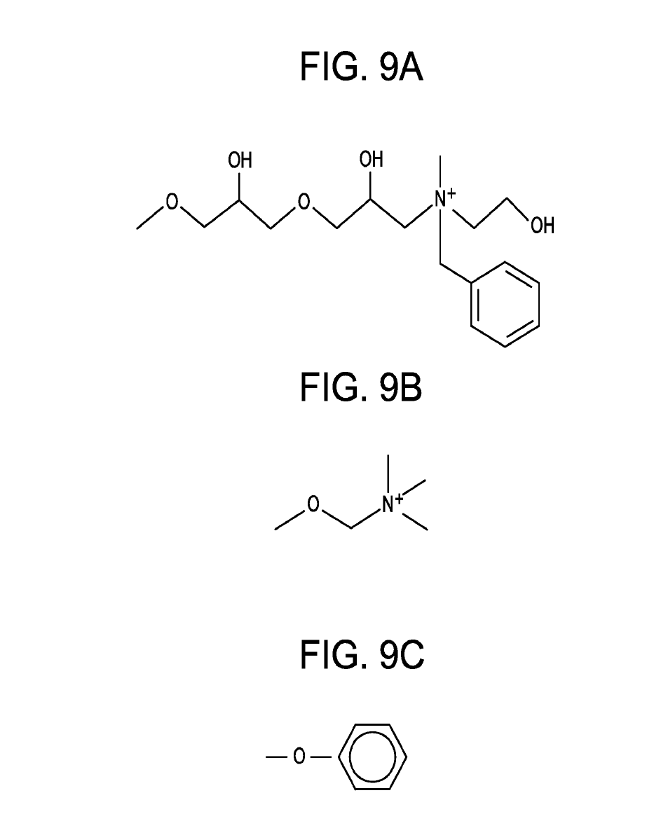

After development of the centerpoint process and the determination of the fragment removal capability of Capto Adhere™, the mechanism of antibody fragmentation product impurity removal was examined. Capto Adhere™ is a mixed mode chromatography resin with anion exchange, hydrophobic interaction, and hydrogen bonding capabilities. To investigate the responsible mechanism, the purification results of the center-point Capto Adhere™ process were first compared to purification results on hydrophobic interaction and anion exchange resins, Capto Phenyl™ and Capto Q™ respectively. The structures of the ligands are shown in FIG. 9. Capto Adhere™ contains a charged amine group (for anion exchange), an aromatic hydrophobic ring (for hydrophobic interactions), and hydroxyls (for hydrogen bonding). Although the Capto Phenyl™ and Capto Q™ have the same backbone at Capto Adhere™, Capto Phenyl™ contains only an aromatic hydrophobic ring and Capto Q™ only has a charged amine group.

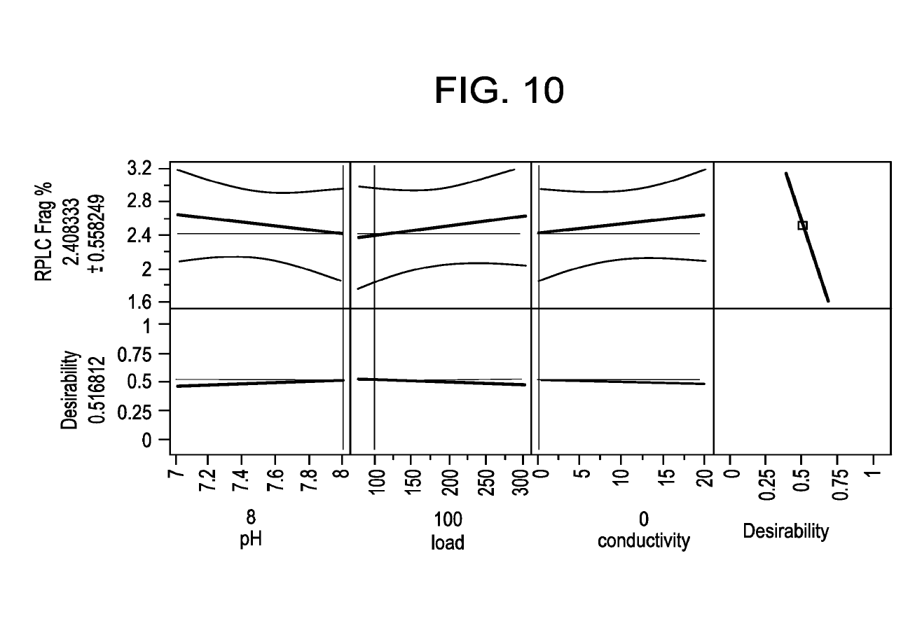

A low pH viral inactivated load was purified by Capto Q™ chromatography in flow through mode under conditions that would be conducive for viral clearance and platform manufacturing. FIG. 10 shows the jmp DOE results for the Capto Q™ experiments. At extreme pH, conductivity and load capacity there is a slight decrease in fragment in the flow through product, from 2.8 to 2.4% RPLC fragment. However, the decrease in fragment failed to match the levels of the Capto Adhere™ products. Over typical manufacturing ranges of pH, conductivity or load capacity, the products purified by Capto Q™ contained similar levels of fragment to each other and to the load material. Based on these results, an anionic mechanism appears to play a minor role, but is not solely responsible for the separation using Capto Adhere™.

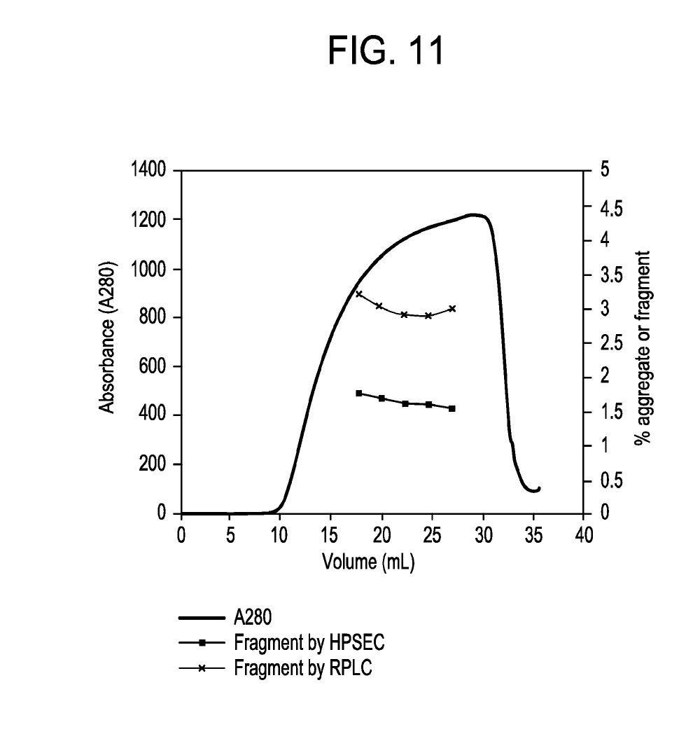

The low pH viral inactivated load was also applied to Capto Phenyl™ resin under flow through conditions at 5 mS/cm, matching the conductivity of the Capto Adhere™ operation. FIG. 11 shows the results of the HIC experiment. HPSEC and RPLC analyses both indicate that the fragment does not interact with the Capto Phenyl™ column, as the fragment levels were consistent across the elution fractionation. HPSEC and RPLC fragment values matched those of the starting materials. The lack of interaction of fragment with the Capto Phenyl™ ligand suggests that hydrophobic interaction is not the only mechanism mediating separation of antibody fragmentation product impurities.

To further study the mechanism of action of antibody fragmentation product impurity removal, resin phase modifiers, ethylene glycol, arginine, and urea, were each separately spiked into load material (Cramer et al. (2011) J Chromatogr A, 1218:7813). The results of the phase modifier experiments are shown in Table 6. Ethylene glycol has been shown to decrease hydrophobic interactions. The addition of this modifier to the load material would disrupt fragment removal on Capto Adhere™ if hydrophobic interactions played a role. In the presence of ethylene glycol, fragment removal by Capto Adhere™ was equivalent to the Capto Adhere™ centerpoint control. Therefore, hydrophobic interactions do not appear to significantly contribute to Capto Adhere™ antibody fragmentation product impurity removal.

TABLE 6

| | HPSEC | | | RPLC |
|---|---|---|---|---|
| | % | % | % | % |
| | monomer | aggregate | fragment | fragment |
| Capto | 97.6 | 0.6 | 1.7 | 2.8 |
| Adhere ™ Load | | | | |
| 0.1M Arginine | 98.5 | 0.5 | 0.9 | 2.3 |
| 2M Urea | 99.4 | 0.6 | 0.0 | 2.3 |
| 20% (v/v) | 99.5 | 0.5 | 0.0 | 1.8 |
| Ethylene Glycol | | | | |

Urea has been shown to impact both hydrophobic and hydrogen bonding interactions. Compared to the ethylene glycol experiment and the centerpoint Capto Adhere™ control, an increase in RPLC fragment to 2.3% was seen in the presence of urea. The urea likely interacted with the fragment's polar side chains or altered the protein's solvation. Since the disruption of hydrophobic interactions by the addition of ethylene glycol did not impact removal of antibody fragmentation product impurities, the urea experiments indicate that hydrogen bonding plays a significant role in the fragment removal.

The results of the arginine phase modifier experiment indicate that ionic interactions may also play a role in the removal of antibody fragmentation product impurities, in addition to hydrogen bonding. Arginine decreases hydrophobic, hydrogen bonding, and ionic interactions. Compared to the urea results, an increase in HPSEC fragment was seen to 0.9%, although the RPLC fragment level remained at 2.3%. The minimal increase in fragment of the arginine experiment compared to the ethylene glycol and urea results demonstrates that, although some ionic interactions contribute to removal of antibody fragmentation product impurities, hydrogen bonding dominates the antibody fragmentation product impurity removal mechanism on Capto Adhere™.

1. A method for separating antibody fragmentation product impurities from a target antibody or a desired antigen-binding fragment thereof, the method comprising:
a) providing a starting material comprising the target antibody or desired antigen-binding fragment, antibody fragmentation product impurities and a loading buffer, wherein the target antibody or desired antigen-binding fragment thereof comprises light chain CDR amino acid sequences selected from LCDR1, LCDR2, LCDR3 of the light chain amino acid sequence shown in SEQ ID NO:2 and heavy chain CDR amino acid sequences selected from HCDR1, HCDR2, HCDR3 of the heavy chain amino acid sequence shown in SEQ ID NO:1;
b) loading the starting material on a mixed mode chromatography column;
c) allowing the material to flow through the column, wherein at least some of the antibody fragmentation product impurities are adsorbed to a stationary phase of the mixed mode chromatography resin and at least some of the target antibody or desired antigen-binding fragment thereof is eluted from the column in one or more eluent fractions; and
d) recovering one or more eluent fractions, wherein one or more eluent fractions are enriched in the target antibody or desired antigen-binding fragment thereof as compared to the starting material.

2-4. (canceled)

5. The method of claim 1, wherein the mixed mode chromatography column comprises a Capto Adhere™ mixed mode chromatography column.

6-9. (canceled)

10. The method of claim 1, wherein one or more eluent fractions comprise between about 1% and about 10% more target antibody or desired antigen-binding fragment thereof than the starting material.

11. The method of claim 1, wherein one or more eluent fractions comprise between about 1% and about 3% more target antibody or desired antigen-binding fragment thereof than the starting material.

12. The method of claim 1, wherein the starting material comprises between about 1% and about 10% antibody fragmentation product impurities.

13. The method of claim 1, wherein the starting material comprises between about 1% and about 3% antibody fragmentation product impurities.

14-16. (canceled)

17. The method of claim 1, wherein the pH of the starting material is between about 5 and about 8.

18-19. (canceled)

20. The method of claim 1, wherein the starting material has a conductivity of between about 0 mS/cm and about 30 mS/cm.

21-24. (canceled)

25. The method of claim 1, wherein the antibody fragmentation product impurities comprise peptide cleavage fragments.

26. The method of claim 25, wherein the antibody fragmentation product impurities are selected from heavy chain fragmentation products, hinge region fragmentation products, light chain fragmentation products, and combinations thereof.

27. The method of claim 1, wherein the antibody fragmentation product impurities comprise antibody reduction fragments.

28. The method of claim 27, wherein the antibody fragmentation product impurities are IgG fragments selected from heavy-heavy-light, heavy-light, light-light, heavy-heavy, heavy, and light.

29-30. (canceled)

31. The method of claim 1, wherein the target antibody or desired antigen-binding fragment thereof comprises a light chain sequence having an amino acid sequence comprising SEQ ID NO:2 and a heavy chain amino acid sequence having an amino acid sequence comprising SEQ ID NO:1.

32-54. (canceled)


SEQUENCE LISTING (2 sequences)

SEQ ID NO: 1 - protein, 117 aa, Artificial Sequence
  Description of Artificial Sequence Synthetic polypeptide
EVQLVQSGAEVKKPGESLKISCKGSGYIFTNYWIAWVRQMPGKCLESMGIIYPGDSDIRYSPSFQGQVTISADKSITTAYLQWSSLKASDTAMYYCARHDIEGFDYWGRGTLVTVSS

SEQ ID NO: 2 - protein, 108 aa, Artificial Sequence [sequence omitted]